(19) United States
(12) Patent Application Publication
(10) Pub. No.: US 2012/0316120 A1
(43) Pub. Date: Dec. 13, 2012

(54) FLUORINE CONTAINING COMPOUNDS AND METHODS OF USE THEREOF

(76) Inventor: Tobias RITTER, Cambridge, MA (US)

(21) Appl. No.: 13/383,055

(22) PCT Filed: Jul. 9, 2010
(86) PCT No.: PCT/US2010/041561

§ 371 Date: Aug. 23, 2012

Related U.S. Application Data
(60) Provisional application No. 61/224,332, filed on Jul. 9, 2009.

(51) Int. Cl. C07K 7/06; A61P 25/36; A61K 38/08

(52) U.S. Cl. 514/17.7; 530/330

(57) ABSTRACT

Fluorinated compounds and methods of making fluorinated compounds are described herein.

### CLAIM OF PRIORITY

This application claims priority from U.S. Ser. No. 61/224,332, filed Jul. 9, 2009 which is incorporated herein by reference in its entirety.

### BACKGROUND OF INVENTION

Functionalized fluorine containing compounds (e.g. aryl fluorides) are often used as pharmaceutical agents. In some embodiments, these products have favorable pharmacological properties such as desirable metabolic stability.

### SUMMARY OF INVENTION

Described herein are methods of making fluorine containing compounds. Also described herein are fluorinated derivatives of compounds (e.g., pharmaceutical agents). Exemplary pharmaceutical agents include a compound described herein or a fluorinated derivative thereof for use as opioid analgesics, and also compounds used to treat opioid dependence, such as an opioid analgesic or opioid dependence agent described herein.

In one aspect, the invention features a method of making a fluorinated compound, such as a compound described herein, using a method described herein.

In one aspect, the invention features a fluorinated enkephalin, for example, a derivative of enkephalin wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F.

In some embodiments, the fluorinated enkephalin has the following formula:

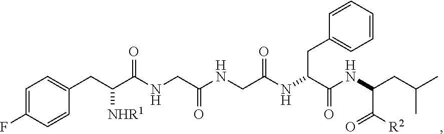

wherein:

R¹ is hydrogen, alkyl or PG¹;

R² is hydroxy, alkoxy, aryloxy, arylalkoxy, —O(PG²), amino, alkylamino, dialkylamino, or —NH(PG¹);

PG¹ is an amino protecting group; and

PG² is a hydroxy protecting group,

or a pharmaceutically acceptable salt thereof.

In some embodiments, R¹ is hydrogen. In some embodiments, R¹ is PG¹ (e.g., tert-butyloxycarbonyl). In some embodiments, R² is hydroxy. In some embodiments, R² is alkoxy (e.g., methoxy). In some embodiments, R² is amino.

In some embodiments, the fluorinated enkephalin has the following formula:

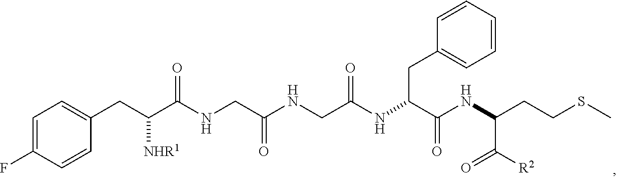

wherein:

R¹ is hydrogen, alkyl or PG¹;

R² is hydroxy, alkoxy, aryloxy, arylalkoxy, —O(PG²), amino, alkylamino, dialkylamino, or —NH(PG¹);

PG¹ is an amino protecting group; and

PG² is a hydroxy protecting group,

or a pharmaceutically acceptable salt thereof.

In some embodiments, R¹ is hydrogen. In some embodiments, R¹ is PG¹ (e.g., tert-butyloxycarbonyl). In some embodiments, R² is hydroxy. In some embodiments, R² is alkoxy (e.g., methoxy). In some embodiments, R² is amino.

In some embodiments, the fluorinated enkephalin is selected from the group consisting of:

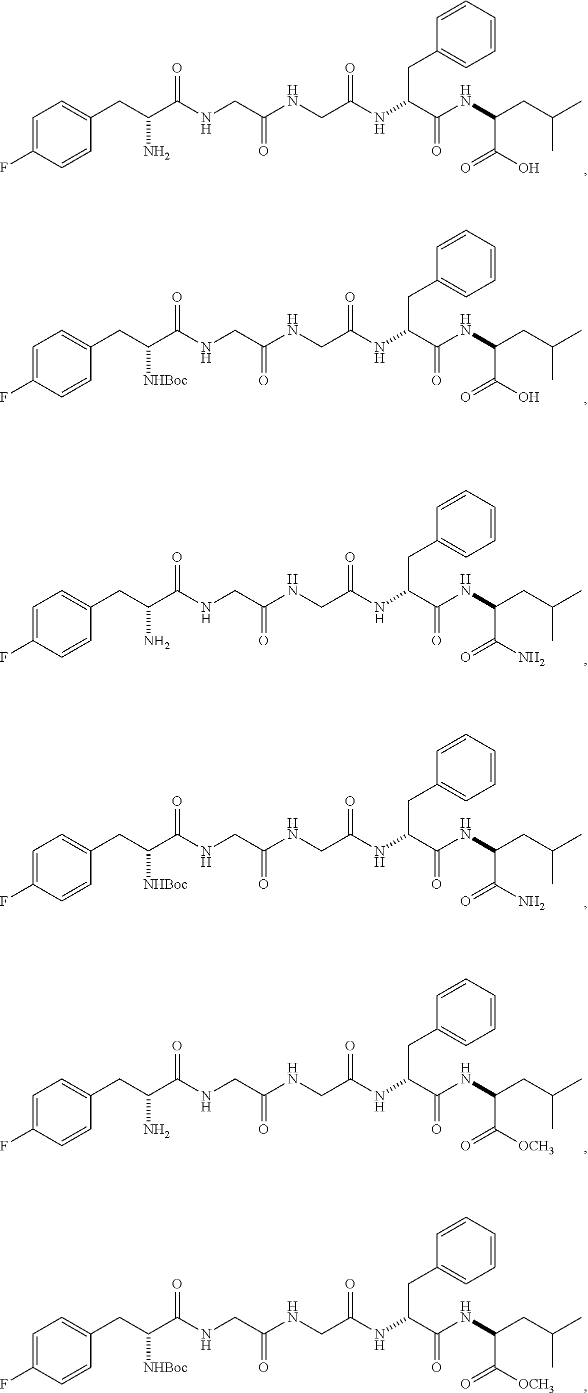

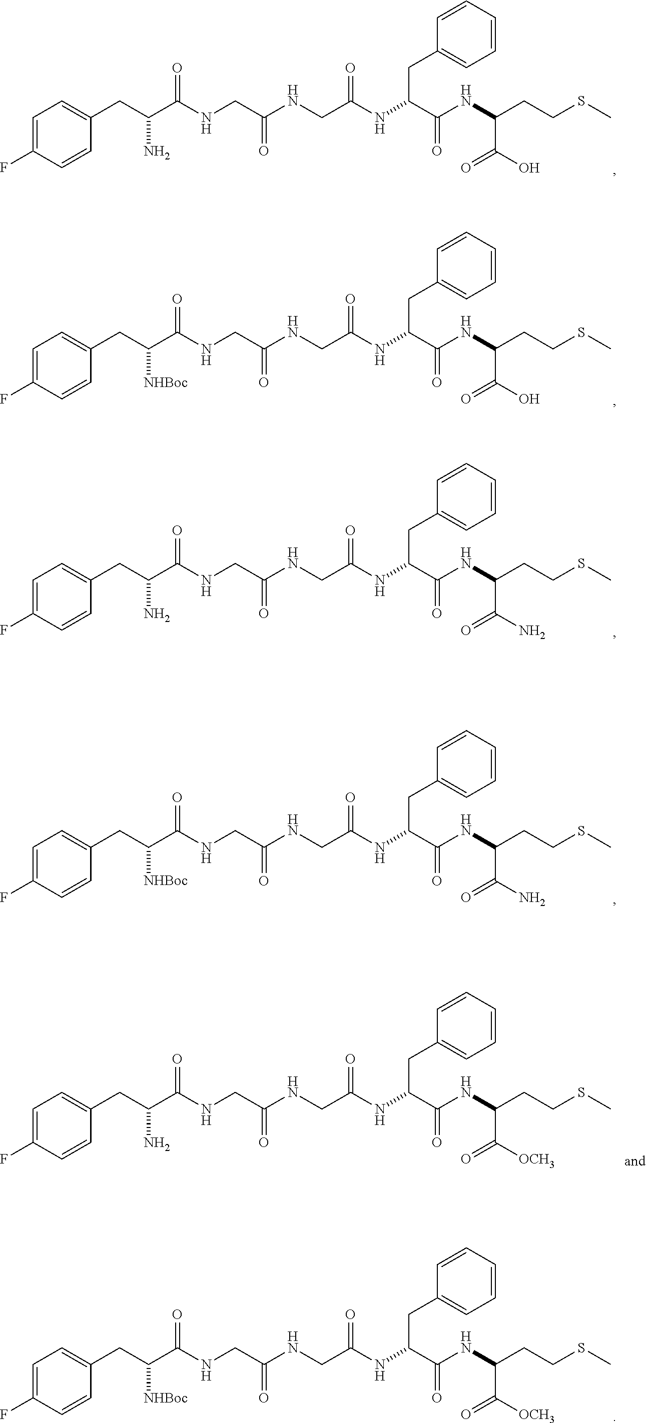

In one aspect, the invention features a method of making a fluorinated enkephalin, for example, a fluorinated enkephalin shown above, using a method described herein.

In one aspect, the invention features a composition comprising a compound described herein (e.g., a pharmaceutical composition comprising a compound described herein).

In one aspect, the invention features a kit comprising a compound or composition described herein.

In some embodiments, a compound described herein can be administered to a subject to treat a disorder described herein, e.g., a disorder that can be treated with an opioid analgesic, or an opioid dependence disorder.

In some embodiments, a compound described herein (e.g., a fluorinated derivative of a pharmaceutical agent) has one or more properties that are superior to a corresponding unfluorinated derivatives of that pharmaceutical agent (e.g., where the corresponding unfluorinated derivative is either without a fluorine in the structure or does not include the same fluorine substitution pattern as the fluorinated derivative described herein). In some embodiments, the improved property is improved metabolic stability, improved penetration across the blood brain barrier, reduced penetration across the blood brain barrier, or improved solubility.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it. The term “haloalkyl” refers to an alkyl in which one or more hydrogen atoms are replaced by halo, and includes alkyl moieties in which all hydrogens have been replaced by halo (e.g., perfluoroalkyl).

The term, “cyano” refers to a —CN radical.

The terms “alkylamino” and “dialkylamino” refer to —NH(alkyl) and —NH(alkyl)₂ radicals respectively. The term “hydroxy” refers to an OH radical. The term “alkoxy” refers to an —O-alkyl radical. The term “mercapto” refers to an SH radical. The term “thioalkoxy” refers to an —S-alkyl radical.

The term “aryl” refers to an aromatic monocyclic, bicyclic, or tricyclic hydrocarbon ring system, wherein any ring atom capable of substitution can be substituted (e.g., by one or more substituents). Examples of aryl moieties include, but are not limited to, phenyl, naphthyl, and anthracenyl.

The term “cycloalkyl” as employed herein includes saturated cyclic, bicyclic, tricyclic, or polycyclic hydrocarbon groups having 3 to 12 carbons. Any ring atom can be substituted (e.g., by one or more substituents). The cycloalkyl groups can contain fused rings. Fused rings are rings that share a common carbon atom. Examples of cycloalkyl moieties include, but are not limited to, cyclopropyl, cyclohexyl, methylcyclohexyl, adamantyl, and norbornyl.

The term “heterocyclyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The heteroatom may optionally be the point of attachment of the heterocyclyl substituent. Any ring atom can be substituted (e.g., by one or more substituents). The heterocyclyl groups can contain fused rings. Fused rings are rings that share a common carbon atom. Examples of heterocyclyl include, but are not limited to, tetrahydrofuranyl, tetrahydropyranyl, piperidinyl, morpholino, pyrrolinyl, pyrimidinyl, quinolinyl, and pyrrolidinyl.

The term “cycloalkenyl” refers to partially unsaturated, nonaromatic, cyclic, bicyclic, tricyclic, or polycyclic hydrocarbon groups having 5 to 12 carbons, preferably 5 to 8 carbons. The unsaturated carbon may optionally be the point of attachment of the cycloalkenyl substituent. Any ring atom can be substituted (e.g., by one or more substituents). The cycloalkenyl groups can contain fused rings. Fused rings are rings that share a common carbon atom. Examples of cycloalkenyl moieties include, but are not limited to, cyclohexenyl, cyclohexadienyl, or norbornenyl.

The term “heterocycloalkenyl” refers to a partially saturated, nonaromatic 5-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The unsaturated carbon or the heteroatom may optionally be the point of attachment of the heterocycloalkenyl substituent. Any ring atom can be substituted (e.g., by one or more substituents). The heterocycloalkenyl groups can contain fused rings. Fused rings are rings that share a common carbon atom. Examples of heterocycloalkenyl include but are not limited to tetrahydropyridyl and dihydropyranyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Any ring atom can be substituted (e.g., by one or more substituents).

The term “acyl” refers to an alkylcarbonyl, cycloalkylcarbonyl, arylcarbonyl, heterocyclylcarbonyl, or heteroarylcarbonyl substituent, any of which may be further substituted (e.g., by one or more substituents).

An “amino protecting group,” as used herein, is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

A “hydroxyl protecting group” as used herein, is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

### DETAILED DESCRIPTION

Compounds

Described herein are fluorinated compounds, e.g., fluorinated derivatives of a pharmaceutical agent. In some embodiments, the compound includes one or more fluorine moieties on an aryl or heteroaryl ring within the pharmaceutical agent.

In some embodiments the compound is a fluorinated derivative of an enkephalin (e.g., an opioid receptor agonist or an opioid receptor antagonist). Exemplary compounds include those in Table 1.

TABLE 1
Exemplary fluorinated compounds.

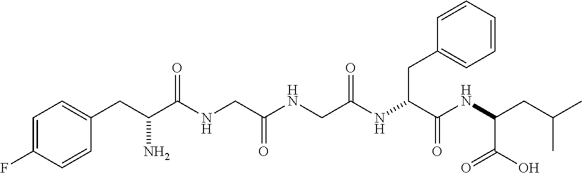

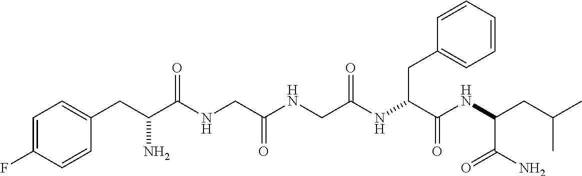

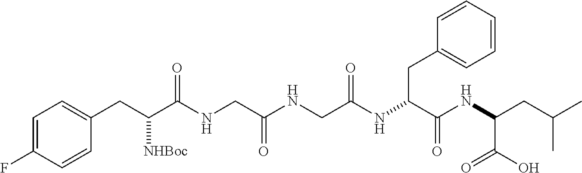

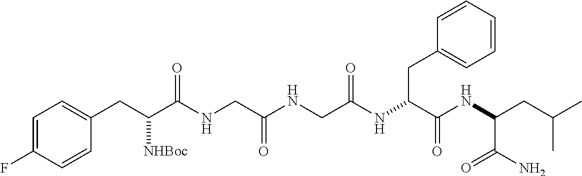

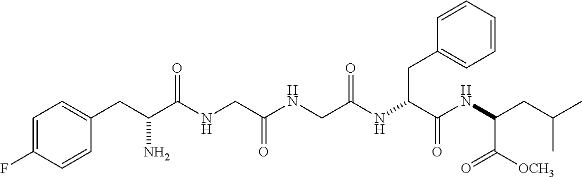

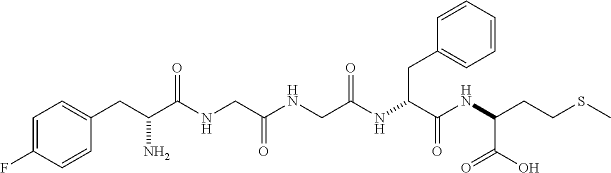

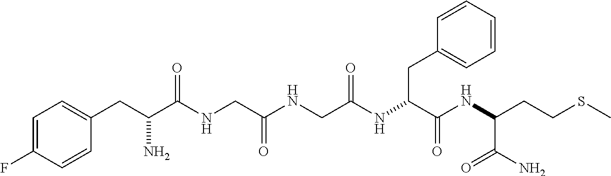

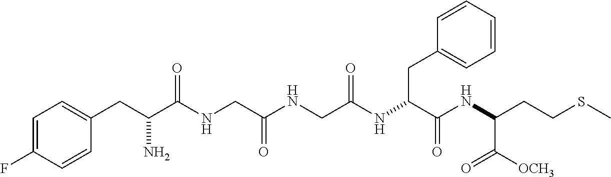

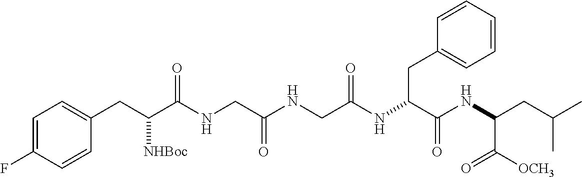

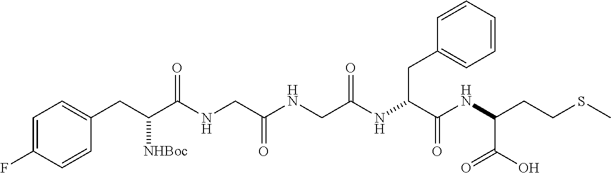

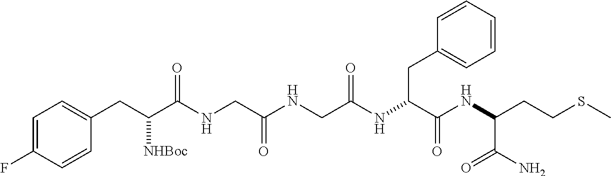

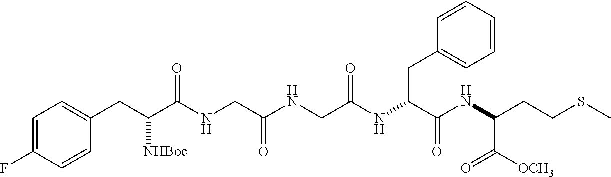

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e.g., carbon-carbon bonds) or substituents that can restrict bond rotation, e.g. restriction resulting from the presence of a ring or double bond. Accordingly, all cis/trans and E/Z isomers are expressly included in the present invention.

The compounds of this invention may also be represented in multiple tautomeric forms. In such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

The compounds of this invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active compounds.

The compounds of this invention may be modified by appending appropriate functionalities to enhance selected biological properties, e.g., targeting to a particular tissue. Such modifications are known in the art and include those which increase biological penetration into a given biological compartment (e.g., blood, lymphatic system, central nervous system), increase oral availability, increase solubility to allow administration by injection, alter metabolism and alter rate of excretion.

In an alternate embodiment, the compounds described herein may be used as platforms or scaffolds that may be utilized in combinatorial chemistry techniques for preparation of derivatives and/or chemical libraries of compounds. Such derivatives and libraries of compounds have biological activity and are useful for identifying and designing compounds possessing a particular activity. Combinatorial techniques suitable for utilizing the compounds described herein are known in the art as exemplified by Obrecht, D. and Villalgrodo, J. M., Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, A. W., Curr. Opin. Chem. Bio., (1997) 1, 60). Thus, one embodiment relates to a method of using the compounds described herein for generating derivatives or chemical libraries comprising: 1) providing a body comprising a plurality of wells; 2) providing one or more compounds identified by methods described herein in each well; 3) providing an additional one or more chemicals in each well; 4) isolating the resulting one or more products from each well. An alternate embodiment relates to a method of using the compounds described herein for generating derivatives or chemical libraries comprising: 1) providing one or more compounds described herein attached to a solid support; 2) treating the one or more compounds identified by methods described herein attached to a solid support with one or more additional chemicals; 3) isolating the resulting one or more products from the solid support. In the methods described above, “tags” or identifier or labeling moieties may be attached to and/or detached from the compounds described herein or their derivatives, to facilitate tracking, identification or isolation of the desired products or their intermediates. Such moieties are known in the art. The chemicals used in the aforementioned methods may include, for example, solvents, reagents, catalysts, protecting group and deprotecting group reagents and the like. Examples of such chemicals are those that appear in the various synthetic and protecting group chemistry texts and treatises referenced herein.

Synthetic Methods

Described herein are methods of making a fluorine-containing compound (e.g., a compound described herein). The compounds described herein can be synthesized via a variety of methods, included Ag or Pd mediated methods. In general, the methods include an organic compound to be fluorinated, a fluorinating agent, and either a silver salt or a palladium complex.

Compounds to be Fluorinated

Exemplary compounds such as a pharmaceutical agent or a precursor thereof or a derivative thereof, include those described herein. The compound may be a small organic molecule or a large organic molecule. A small organic molecule includes any molecule having a molecular weight of less than 1000 g/mol, of less than 900 g/mol, of less than 800 g/mol, of less than 700 g/mol, of less than 600 g/mol, of less than 500 g/mol, of less than 400 g/mol, of less than 300 g/mol, of less than 200 g/mol or of less than 100 g/mol. A large organic molecule include any molecule of between 1000 g/mol to 5000 g/mol, of between 1000 g/mol to 4000 g/mol, of between 1000 g/mol to 3000 g/mol, of between 1000 g/mol to 2000 g/mol, or of between 1000 g/mol to 1500 g/mol. Organic compounds include aryl compounds, heteroaryl compounds, carbocyclic compounds, heterocyclic compounds, aliphatic compounds, heteroaliphatic compounds. In a preferred embodiment, the organic compound is an aryl compound (e.g., a phenyl compound), or a heteroaryl compound (e.g. a quinolyl or indolyl compound). The compound may be a peptide (a short polymer formed from the linking, in a defined order, of α-amino acids via amide bonds). Exemplary peptides include compounds having 2, 3, 4, 5, 6, 7, 8, 9, 10, etc. amino acids (e.g., a peptide such as an enkephalin).

In some embodiments, the compound contains a chiral center. In some embodiments, the compound is further substituted with one or more functional groups (e.g., alcohols, aldehydes, ketones, alkenes, alkoxy groups, cyano groups, amides and N-oxides). In some embodiments, the functional groups are unprotected. In some embodiments, the compound is a precursor of a pharmaceutically acceptable compound.

In some embodiments, the compounds disclosed herein can be prepared on a solid support. The term “solid support” refers a material to which a compound is attached to facilitate identification, isolation, purification, or chemical reaction selectivity of the compound. Such materials are known in the art and include, for example, beads, pellets, disks, fibers, gels, or particles such as cellulose beads, pore-glass beads, silica gels, polystyrene beads optionally cross-linked with divinylbenzene and optionally grafted with polyethylene glycol, poly-acrylamide beads, latex beads, dimethylacrylamide beads optionally cross-linked with N,N′-bis-acryloyl ethylene diamine, glass particles coated with hydrophobic polymer, and material having a rigid or semi-rigid surface. The solid supports optionally have functional groups such as amino, hydroxy, carboxy, or halo groups, (see, Obrecht, D. and Villalgrodo, J. M., Solid-Supported Combinatorial and Parallel Synthesis of Small-Molecular-Weight Compound Libraries, Pergamon-Elsevier Science Limited (1998)), and include those useful in techniques such as the “split and pool” or “parallel” synthesis techniques, solid-phase and solution-phase techniques, and encoding techniques (see, for example, Czarnik, A. W., Curr. Opin. Chem. Bio., (1997) 1, 60).

Fluorinating Agents

As generally described above, the process utilizes a fluorinating agent. In certain embodiments, the fluorinating agent is an electrophilic fluorinating agent. In certain embodiments, the fluorinating agent is commercially available. In certain embodiments, the electrophilic fluorinating agent is also an inorganic fluorinating agent. Exemplary electrophilic fluorinating agents include, but are not limited to, N-fluoropyridinium triflate, N-fluoro-2,4,6-trimethylpyridinium triflate, N-fluoro-2,4,6-trimethylpyridinium tetrafluoroborate, N-fluoro-2,6-dichloropyridinium tetrafluoroborate, N-fluoro-2,6-dichloropyridinium triflate, N-fluoropyridinium pyridine heptafluorodiborate, N-fluoropyridinium tetrafluoroborate, N-fluoropyridinium triflate, an N-fluoroarylsulfonimide (e.g., N-fluorobenzenesulfonimide), N-chloromethyl-N′-fluorotriethylenediammonium bis(tetrafluoroborate) (Selectfluor®), N-chloromethyl-N′-fluorotriethylenediammonium bis(hexafluorophosphate), N-chloromethyl-N′-fluorotriethylenediammonium bis(triflate) and XeF₂ In certain embodiments, the fluorinating agent is Selectfluor®. In certain embodiments, the fluorinating agent is N-fluoropyridinium triflate. In certain embodiments, the fluorinating agent is N-fluoro-2,4,6-trimethylpyridinium triflate. In certain embodiments, the fluorinating agent is N-fluoro-2,4,6-trimethylpyridinium tetrafluoroborate. In certain embodiments, the fluorinating agent is N-fluoro-benzenesulfonimide. In certain embodiments, the fluorinating agent is xenon difluoride.

The fluorinating agent may be enriched with a particular isotope of fluorine. In certain embodiments, the fluorinating agent is labeled with ¹⁹F (i.e., transfers an ¹⁹F fluorine substituent to the organic compound). In certain embodiments, reaction of the ¹⁹F fluorinating agent in the inventive process provides a fluorinated ¹⁹F-labeled organic compound.

In certain embodiments, the fluorinating agent is labeled with ¹⁸F (i.e., transfers an ¹⁸F fluorine substituent to the organic compound). In certain embodiments, reaction of the ¹⁸F fluorinating agent in the inventive process provides a fluorinated ¹⁸F-labeled organic compound.

However, in certain embodiments, the fluorinating agent is labeled with a mixture of ¹⁸F and ¹⁹F. In certain embodiments, reaction of the mixture of ¹⁹F and ¹⁸F fluorinating agent in the inventive process provides a mixture of fluorinated ¹⁹F-labeled organic compound and fluorinated ¹⁸F-labeled organic compound.

Any of the above fluorinated agents may be labeled as ¹⁹F or ¹⁸F.

For example, in certain embodiments, the fluorinating agent is ¹⁹F-labeled N-(chloromethyl)-N′-fluorotriethylenediamine bis(tetrafluoroborate) (Selectfluor®) or ¹⁹F-labeled XeF₂. In certain embodiments, the fluorinating agent is ¹⁹F-labeled N-(chloromethyl)-N′-fluorotriethylenediamine bis(tetrafluoroborate) (Selectfluor®). In certain embodiments, the fluorinating agent is ¹⁹F-labeled XeF₂.

In certain embodiments, the fluorinating agent is ¹⁸F-labeled N-(chloromethyl)-N′-fluorotriethylenediamine bis(tetrafluoroborate) (Selectfluor®) or ¹⁸F-labeled XeF₂. In certain embodiments, the fluorinating agent is ¹⁸F-labeled N-(chloromethyl)-N′-fluorotriethylenediamine bis(tetrafluoroborate) (Selectfluor®). In certain embodiments, the fluorinating agent is ¹⁸F-labeled XeF₂.

Exemplary methods include the following.

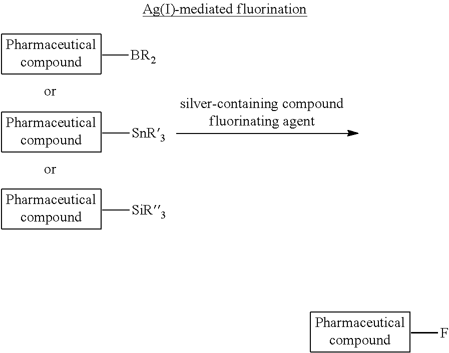

Upon reaction of an organic compound comprising an organostannane, a boron substituent or a silane substituent, with a silver-containing compound and a fluorinating agent, the method provides a fluorinated organic compound in which the organostannane, boron substituent or silane substituent is replaced with a fluorine substituent. In some embodiments, the organostannane, boron substituent or silane substituent is attached to an aryl or heteroaryl moiety of the organic compound. For examples, see Schemes 1-5.

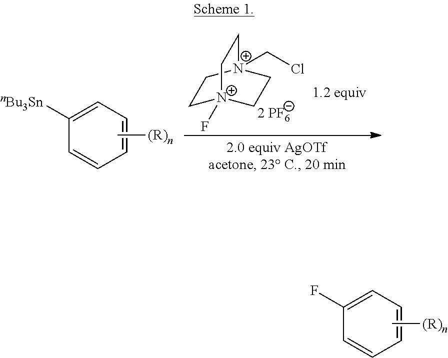

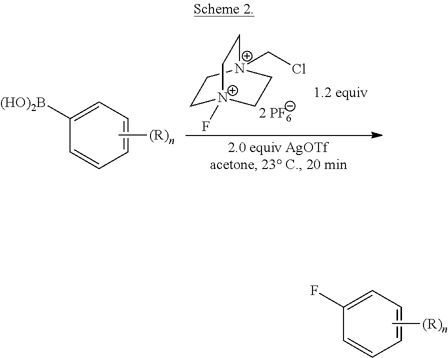

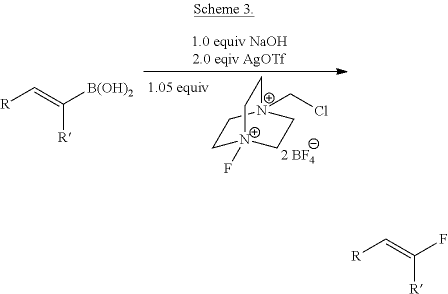

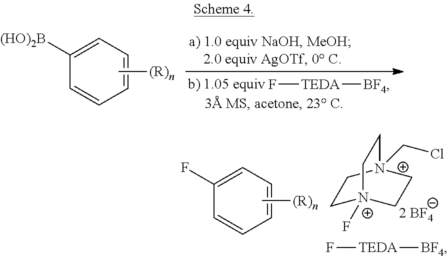

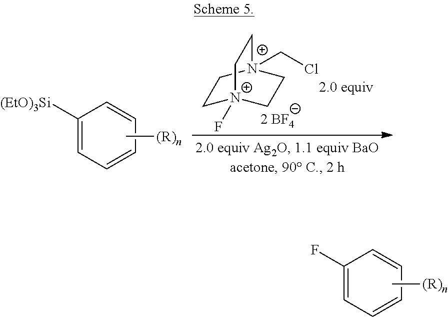

In some embodiments, the method uses a catalytic amount of silver. Exemplary methods of fluorinating a compound using Ag are described in U.S. Provisional Patent Application 61/177,907, filed May 14, 2009, which is incorporated herein by reference in its entirety.

Boron Substituents

In some embodiments, the organic compound (e.g., pharmaceutical agent or derivative thereof) comprises a boron substituent, e.g., a group of the formulae:

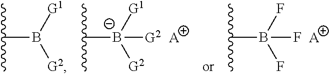

wherein G¹, G² and G³ are, independently, —OH, —OR, or —R, wherein each R is, independently, optionally substituted aliphatic, optionally substituted heteroaliphatic, optionally substituted aryl, or optionally substituted heteroaryl, or G¹ and G² are joined to form an optionally substituted 5- to 8-membered ring having at least one O atom directly attached to B, wherein the ring is comprised of carbon atoms and optionally one or more additional heteroatoms independently selected from the group consisting of N, S, and O. A⁺ may be a metal cation or ammonium.

As used herein, a boron substituent is intended to encompass free boronic acid substituents (i.e., wherein G¹ and G² are both —OH) and oligomeric anhydrides thereof (including dimers, trimers, and tetramers, and mixtures thereof), boronic ester substituents (i.e., wherein G¹ is —OH or —OR and G² is —OR), borinic acid substituents (i.e., wherein G¹ is —OH and G² is —R), borinic ester substituents (i.e., wherein G¹ is —OR and G² is —R), trihydroxoborates (i.e., wherein G¹, G² and G³ are all —OH), and trialkoxyborates (i.e., wherein G′, G² and G³ are all —OR, e.g., —OCH₃).

In some embodiments, G¹ and G² are joined to form a 5-membered ring. Exemplary 5-membered rings include:

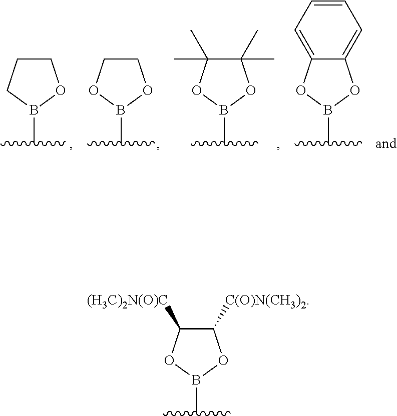

In some embodiments, G¹ and G² are joined to form a 6-membered ring. Exemplary 6-membered rings include:

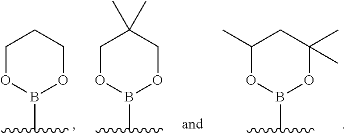

In some embodiments, G¹ and G² are joined to form an 8-membered ring. Exemplary 8-membered rings include:

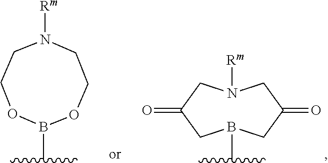

wherein R^{m} is hydrogen, a suitable amino protecting group, or an optionally substituted aliphatic, optionally substituted heteroaliphatic, optionally substituted aryl, or optionally substituted heteroaryl group.

Furthermore, as used herein, a boron substituent is also intended to encompass a trifluoroborate substituent. For example, in some embodiments, a boron substituent is a group of the formula:

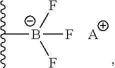

wherein A^{⊕} is a metal cation or ammonium.

Furthermore, as used herein, a boron substituent is also intended to encompass trihydroxy- and trialkoxy borates. For example, in some embodiments, a boron substituent is a group of the formulae:

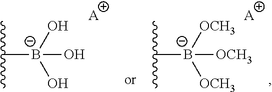

wherein A^{⊕} is a metal cation or ammonium.

Exemplary metal cations include lithium, sodium, potassium, magnesium, and calcium cations. In some embodiments, the metal cation is a potassium cation.

An organic compound comprising a boron substituent may be obtained via a variety of known methods. For example, a halogen-containing precursor may be reacted with a boron-containing compound to generate the organic compound comprising a boron substituent. An unactivated C—H bond may also be borylated, for example, using a suitable catalyst.

Silane Substituents

Methods of fluorinating an organic compound are described herein. In some embodiments, the organic compound comprises a silane substituent. The silane substituent may be a trialkoxysilane, e.g., trimethoxysilane or triethoxysilane. The silane substituent may be a trihydroxysilane.

An organic compound comprising a silane substituent may be obtained via a variety of known methods. For example, a Grignard-containing precursor may be reacted with a silicon-containing compound (e.g., a tetraalkoxysilane) to generate the organic compound comprising a silane substituent. In another example, a halogen-containing precursor or a triflyl-containing precursor may be reacted with a silicon-containing compound (e.g., a tetraalkoxysilane) in the presence of a suitable catalyst (e.g., a Pd⁰ or R^{I} catalyst) to generate the organic compound comprising a silane substituent.

Organostannanes

Methods of fluorinating an organic compound are described herein. In some embodiments, the organic compound comprises an organostannane. The organostannane may be a trialkylstannane, e.g., trimethylstannane or tributylstannane.

Silver-Containing Compounds

The methods described herein generally include a silver-containing compound. The silver-containing compound may be a silver complex or a silver salt, e.g., a silver(I) salt. Exemplary silver salts include silver(I) salts such as silver(I) fluoride, silver(I) acetate, silver(I) tetrafluoroborate, silver(I) perchlorate, silver(I) nitrate, silver(I) carbonate, silver(I) cyanide, silver(I) benzoate, silver(I) triflate, silver(I) hexafluorophosphate, silver(I) hexafluoroantimonate, silver(I) oxide, silver(I) nitrite and silver(I) phosphate. In preferred embodiments, the silver salt is silver(I) triflate or silver(I) oxide.

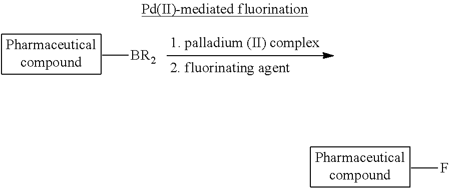

Upon reaction of an organic compound comprising a boron substituent with a palladium(II) complex and a fluorinating agent, the method provides a fluorinated organic compound in which the boron substituent is replaced with a fluorine substituent. In some embodiments, the boron substituent is attached to an aryl or heteroaryl moiety of the organic compound. For example, see Scheme 6.

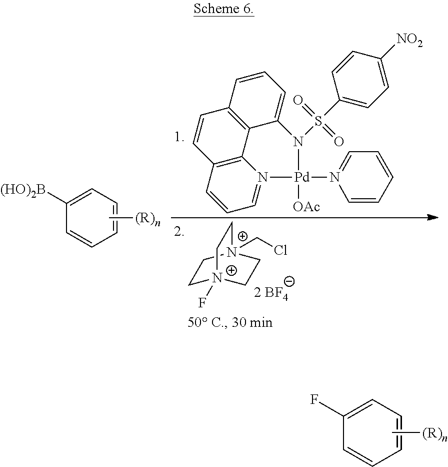

Exemplary methods of fluorinating a compound using a Pd(II) complex are described in PCT Application PCT/US2009/032855, filed Feb. 2, 2009, which is incorporated herein by reference in its entirety.

### Palladium (II) Complexes

In certain embodiments, a stoichiometric amount of the palladium (II) complex is used.

In certain embodiments, the palladium (II) complex comprises a bidentate ligand. In certain embodiments, the palladium (II) complex comprises a tridentate ligand.

In certain embodiments, the palladium (II) complex is crystalline. Alternatively, in certain embodiments, the palladium (II) complex is amorphous.

In certain embodiments, the palladium (II) complex is not a salt. Alternatively, in certain embodiments, the palladium (II) complex is a salt. For example, in certain embodiments, the palladium (II) complex is a salt of tetrafluoroborate (BF₄⁻), tetraphenylborate (BPh₄⁻), phorphorous hexafluoride (PF₆⁻), BArF-tetrakis(pentafluorophenyl)borate, antimohexafluoride (SbF₆⁻), or trifluoromethansulfonate (triflate, CF₃SO₃⁻). In certain embodiments, the palladium (II) complex is a salt of tetrafluoroborate (BF₄⁻).

In certain embodiments, the palladium (II) complex is a palladium (II) dimer complex.

In certain embodiments, the palladium (II) complex is generated in situ from a complex in the 0 oxidation state (i.e., a “palladium (0) complex”) and one or more ligands.

Exemplary ligands include, but are not limited to, halogens (e.g., iodide, bromide, chloride, fluoride), solvents (e.g., hydroxide, water, ammonia, acetonitrile, dimethylsulfoxide, dimethylformamide, dimethylacetamide), sulfide, cyanide, carbon monoxide, thiocyanate, isothiocyanate, nitrate, nitrite, azide, oxalate, olefins (e.g., dibenzylidineacetone (dba)), optionally substituted pyridines (py) (e.g., 2,2′,5′,2-terpyridine (terpy), bipyridine (bipy) and other pyridine ligands as described herein), optionally substituted aryl (e.g., phenyl (Ph), phenanthroline (phen), biphenyl), phosphines (e.g., triphenylphosphine (PPh₃), 1,2-bis(diphenylphosphino)ethane (dppe), tricyclohexylphosphine (PCy₃), tri(o-tolyl)phosphine (P(o-tol)₃), tris(2-diphenylphosphineethyl)amine (np3)), amino ligands (e.g., ethylenediamine (en), diethylenetriamine (dien), tris(2-aminoethyl)amine (tren), triethylenetetramine (trien), ethylenediaminetetraacetate (EDTA)), acyloxy ligands (e.g., acetylaceonate (acac), O-acetate (—OAc)), and alkyloxy ligands (e.g., —OMe, OiPr, OtBu).

As one of ordinary skill in the art would understand, the ligands are chosen to satisfy the valency of palladium. Thus, in certain embodiments, the ligands are chosen to satisfy the valency of a palladium complex as +2.

Exemplary palladium (II) complexes include, but are not limited to, palladium (II) bromide, palladium (II) chloride, palladium (II) iodide, palladium (II) fluoride, palladium (II) acetate, palladium (II) acetylacetonate, palladium (II) oxide, palladium (II) cyanide, palladium (II) sulfide, palladium (II) sulfate, palladium (II) 2,4-pentanedionate, allyl palladium (II) chloride dimer, bis(acetonitrile)dichloropalladium (II), trans-bis(benzonitrile)dichloropalladium (II), and trichloro-bis(triphenylphosphine)palladium (II).

Exemplary palladium (0) complexes include, but are not limited to, Pd₂dba₃, Pd₂ dba₃-CHCl₃, and tetrakis(triphenylphosphine)palladium (0).

Other exemplary ligands are provided as groups R^{L1} and R^{L2}, described below and herein. Furthermore, other exemplary bidentate and tridentate palladium (II) complexes are provided in the following formulae, described below and herein.

For example, in certain embodiments, the palladium (II) complex comprises a bidentate or tridentate ligand to provide a complex of the formula (I):

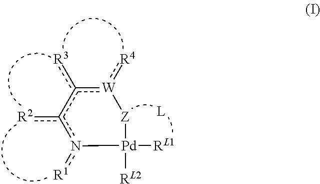

wherein:

Pd represents palladium of valency of +2;

R^{L1} and R^{L2} are, independently, optionally substituted aliphatic, optionally substituted heteroaliphatic, optionally substituted aryl, optionally substituted heteroaryl, halogen, —OR^{a}, —SR^{b}, —N(R^{c})₂, —N(R^{c})₃, or —P(R^{x})₃,

wherein each instance of R^{a} is, independently, hydrogen, an optionally substituted aliphatic, optionally substituted heteroaliphatic, optionally substituted aryl, optionally substituted heteroaryl, —C(═O)R^{a1}, —C(═O)OR^{a2}, —C(═O)N(R^{a3})₂, —C(═NR^{a3})R^{a3}, —C(═NR^{a3})OR^{a1}, —C(═NR^{a3})N(R^{a3})₂, —S(O)₂R^{a1}, —S(O)R^{a1}, or a suitable hydroxyl protecting group, wherein R^{a1} is an optionally substituted aliphatic, optionally substituted heteroaliphatic, optionally substituted aryl or optionally substituted heteroaryl group; wherein R^{a2} is an optionally substituted aliphatic, optionally substituted heteroaliphatic, optionally substituted aryl, optionally substituted heteroaryl group, or a suitable hydroxyl protecting group; wherein R^{a3} is an optionally substituted aliphatic, optionally substituted heteroaliphatic, optionally substituted aryl, optionally substituted heteroaryl group, or a suitable amino protecting group, or two R^{a3} groups are joined to form an optionally substituted heterocyclic or heteroaryl ring;

wherein each instance of R^{b} is, independently, an optionally substituted aliphatic, optionally substituted heteroaliphatic, optionally substituted aryl, optionally substituted heteroaryl, —C(═O)R^{b1}, —C(═O)OR^{b2}, —C(═O)N(R^{b3})₂, —C(═NR^{b3})R^{b3}, —C(═NR^{b3})OR^{b1}, —C(═NR^{a3})N(R^{b3})₂, or a suitable thiol protecting group, wherein R^{b1} is an optionally substituted aliphatic, optionally substituted heteroaliphatic, optionally substituted aryl or optionally substituted heteroaryl group; wherein R^{b2} is an optionally substituted aliphatic, optionally substituted heteroaliphatic, optionally substituted aryl, optionally substituted heteroaryl group, or a suitable hydroxyl protecting group; wherein R^{b3} is an optionally substituted aliphatic, optionally substituted heteroaliphatic, optionally substituted aryl, optionally substituted heteroaryl group, or a suitable amino protecting group, or two R^{b3} groups are joined to form an optionally substituted heterocyclic or heteroaryl ring;

wherein each instance of R^{c} is, independently, hydrogen, an optionally substituted aliphatic, optionally substituted heteroaliphatic, optionally substituted aryl, optionally substituted heteroaryl, —C(═O)R^{c1}, —C(═O)OR^{c2}, —C(═O)N(R^{c3})₂, —C(═NR^{c3})R^{c3}, —C(═NR^{c3})OR^{c1}, —C(═NR^{c3})N(R^{c3})₂, —S(O)₂R^{c1}, —S(O)R^{c1}, or a suitable amino protecting group, or two R^{c} groups are joined to form an optionally substituted heterocyclic or heteroaryl ring or the group ≡C(R^{c1}), wherein R^{c1} is an optionally substituted aliphatic, optionally substituted heteroaliphatic, optionally substituted aryl or optionally substituted heteroaryl group; wherein R^{c2} is an optionally substituted aliphatic, optionally substituted heteroaliphatic, optionally substituted aryl, optionally substituted heteroaryl group, or a suitable hydroxyl protecting group; wherein R^{c3} is an optionally substituted aliphatic, optionally substituted heteroaliphatic, optionally substituted aryl, optionally substituted heteroaryl group, or a suitable amino protecting group, or two R^{c3} groups are joined to form an optionally substituted heterocyclic or heteroaryl ring;

wherein each instance of R^{x} is, independently, hydrogen, an optionally substituted aliphatic, optionally substituted alkoxy, optionally substituted heteroaliphatic, optionally substituted aryloxy, optionally substituted heteroaryloxy, optionally substituted aryl, or optionally substituted heteroaryl group;

when W is —C— or —C(R^{d})— then:
(i) Z is a bond, —O—, —S—, —C(R^{d})₂—, —C(R^{d})═C(R^{d})—, —C(R^{d})═N—, or —N(R^{e})—;

or
(ii) Z is —N— joined via a linker group -L- to the group R^{L1} to form a 5- to 7-membered palladacycle, wherein -L- is selected from absent, —C(═O)—, —C(═O)O—, —C(═O)N(R^{e3})—, —C(═NR^{e3})—, —C(═NR^{e3})—, —C(═NR^{e3})N(R^{e3})—, —S(O)₂—, or —S(O)— and R^{L1} is an optionally substituted aryl, optionally substituted heteroaryl, or an —N(R^{c})₂ group wherein two R^{c} groups are joined to form an optionally substituted heterocyclic or heteroaryl ring;

or
(iii) Z is —N—S(O)₂—R^{e3} and the linker group -L- is absent;

or

when W is —N— or —N(R^{e})—, then Z is a bond, —C(R^{d})₂, —C(R^{d})═C(R^{d})—, or —C(R^{d})═N—;

or

when W is —SO₂— or ═N—, then R₄ is absent;

wherein each instance of R^{d} is, independently, hydrogen, or an optionally substituted aliphatic, optionally substituted heteroaliphatic, optionally substituted aryl, or optionally substituted heteroaryl group; and

each instance of R^{e} is, independently, hydrogen, an optionally substituted aliphatic, optionally substituted heteroaliphatic, optionally substituted aryl, optionally substituted heteroaryl, —C(═O)R^{e1}, —C(═O)OR^{e2}, —C(═O)N(R^{e3})₂, —C(═NR^{e3})R^{e1}, —C(═NR^{e3})OR^{e2}, —C(═NR^{e3})N(R^{e3})₂, —S(O)₂R^{e1}, —S(O)R^{e1}, a suitable amino protecting group, wherein R^{e1} is an optionally substituted aliphatic, optionally substituted heteroaliphatic, optionally substituted aryl or optionally substituted heteroaryl group; wherein R^{e2} is an optionally substituted aliphatic, optionally substituted heteroaliphatic, optionally substituted aryl, optionally substituted heteroaryl group, or a suitable hydroxyl protecting group; wherein R^{e3} is an optionally substituted aliphatic, optionally substituted heteroaliphatic, optionally substituted aryl, optionally substituted heteroaryl group, or a suitable amino protecting group, or two R^{e3} groups are joined to form an optionally substituted heterocyclic or heteroaryl ring;

R¹, R², R³ and R⁴ are, independently, an optionally substituted aliphatic, optionally substituted heteroaliphatic, optionally substituted aryl, optionally substituted heteroaryl group,

R¹ and R² are optionally joined to form an optionally substituted 5- to 7-membered heteroaryl, aryl, heterocyclic or carbocyclic ring;

R² and R³ are optionally joined to form an optionally substituted 5- to 7-membered heteroaryl, aryl, heterocyclic or carbocyclic ring;

R³ and R⁴ are optionally joined to form an optionally substituted 5- to 7-membered heteroaryl, aryl, heterocyclic or carbocyclic ring,

wherein the each of curved dotted lines

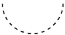

independently represents optional joining of an optionally substituted 5- to 7-membered ring, and

wherein [symbol] represents a single or double bond.

In certain embodiments, R¹ and R² are joined to form an optionally substituted 5- to 6-membered heteroaryl, aryl, heterocyclic or carbocyclic ring. In certain embodiments, R¹ and R² are joined to form an optionally substituted 5-membered heteroaryl, aryl, heterocyclic or carbocyclic ring. In certain embodiments, R¹ and R² are joined to form an optionally substituted 6-membered heteroaryl, aryl, heterocyclic or carbocyclic ring.

In certain embodiments, R² and R³ are joined to form an optionally substituted 5- to 6-membered heteroaryl, aryl, heterocyclic or carbocyclic ring. In certain embodiments, R² and R³ are joined to form an optionally substituted 5-membered heteroaryl, aryl, heterocyclic or carbocyclic ring. In certain embodiments, R² and R³ are joined to form an optionally substituted 6-membered heteroaryl, aryl, heterocyclic or carbocyclic ring.

In certain embodiments, R³ and R⁴ are joined to form an optionally substituted 5- to 6-membered heteroaryl, aryl, heterocyclic or carbocyclic ring. In certain embodiments, R³ and R⁴ are joined to form an optionally substituted 5-membered heteroaryl, aryl, heterocyclic or carbocyclic ring. In certain embodiments, R³ and R⁴ are joined to form an optionally substituted 6-membered heteroaryl, aryl, heterocyclic or carbocyclic ring.

Any of the optionally substituted 5- to 6-membered heteroaryl, aryl, heterocyclic or carbocyclic rings formed by joining R¹ and R², R² and R³ and/or R³ and R⁴ can be, for example, an optionally substituted 5- to 6-membered heteroaryl, an optionally substituted 6-membered aryl, an optionally substituted 5- to 6-membered heterocyclic or an optionally substituted 5- to 6-membered carbocyclic ring.

Exemplary 5-membered heteroaryl rings include, but are not limited to, optionally substituted pyrrolyl, optionally substituted pyrazolyl, optionally substituted imidazolyl, optionally substituted triazolyl or optionally substituted tetrazolyl, optionally substituted thiazolyl, optionally substituted isothazolyl, optionally substituted thiadiazolyl, optionally substituted oxazolyl, optionally substituted isooxazolyl, optionally substituted oxadiaziolyl or optionally substituted oxadiaziolyl ring.

Exemplary 6-membered heteroaryl rings include, but are not limited to, optionally substituted pyridinyl, optionally substituted pyrimidinyl, optionally substituted pyrazinyl, optionally substituted pyridazinyl, optionally substituted triazinyl or optionally substituted tetrazinyl ring.

Exemplary 5-membered heterocyclic rings include, but are not limited to, optionally substituted pyrrolidinyl, optionally substituted tetrahydrofuranyl, optionally substituted tetrahydrothiophenyl, and optionally substituted 1,3 dithiolanyl.

Exemplary 6-membered heterocyclic rings include, but are not limited to, optionally substituted piperidinyl, optionally substituted piperazinyl, optionally substituted morpholinyl, optionally substituted tetrahydropyranyl and optionally substituted dioxanyl.

Exemplary 5-membered carbocyclic rings include, but are not limited to, optionally substituted cyclopentyl and optionally substituted cyclopentenyl.

Exemplary 6-membered carbocyclic rings include, but are not limited to, optionally substituted cyclohexyl and optionally substituted cyclohexenyl.

In certain embodiments, R² and R³ are not joined together to form a cyclic structure.

In certain embodiments, R³ and R⁴ are not joined together to form a cyclic structure.

In certain embodiments, both R¹ and R² and R² and R³ are joined to form rings, but R³ and R⁴ are not joined together to form a cyclic structure.

In certain embodiments, both R¹ and R² and R³ and R⁴ joined to form rings, but R² and R³ are not joined together to form a cyclic structure.

In certain embodiments, both R² and R³ and R³ and R⁴ are joined to form rings, but R¹ and R² are not joined together to form a cyclic structure.

Palladium (II) Complexes with Bidentate Ligand

In certain embodiments, Z is not joined via a linker group -L- to the group R^{L1} form a 5- to 7-membered palladacycle.

For example, in certain embodiments, the palladium (II) complex comprises a bidentate ligand. In certain embodiments, the palladium (II) complex is of the formula (I-a):

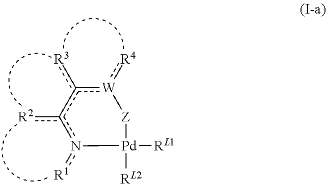

wherein Pd, [symbol] ,

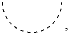

W, R^{L1}, R^{L2}, Z, R¹, R², R³ and R⁴ are as defined above and herein.

In certain embodiments, R¹ and R² are joined to form an optionally substituted 6-membered pyridinyl ring to provide a palladium (II) complex of the formula (I-b):

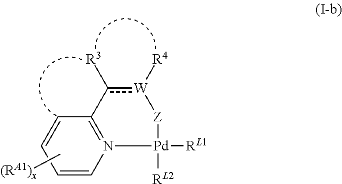

wherein

Pd, [symbol] ,

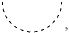

W, R^{L1}, R^{L2}, Z, R³, and R⁴ are as defined above and herein;

each instance of R^{A1} is, independently, hydrogen, halogen, optionally substituted aliphatic, optionally substituted heteroaliphatic, optionally substituted aryl, optionally substituted heteroaryl, —CN, —NO₂, —NC, —OR^{A1a}, —SR^{A1b}, —N(R^{A1c})₂, —C(═O)R^{A1d}, —C(═O)OR^{A1a}, —C(═O)N(R^{A1c})₂, —C(═NR^{A1c})R^{A1d}, —C(═NR^{A1c})OR^{A1a}, —C(═NR^{A1c})N(R^{A1c})₂, —S(O)₂R^{A1d}, —S(O)R^{A1d}, or two R^{A1} groups adjacent to each other are joined to form a 5- to 6-membered aryl, heteroaryl, heterocyclic or carbocyclic ring, wherein R^{A1a} is hydrogen, an optionally substituted aliphatic, optionally substituted heteroaliphatic, optionally substituted aryl, optionally substituted heteroaryl or a suitable hydroxyl protecting group; wherein R^{A1b} is hydrogen, an optionally substituted aliphatic, optionally substituted heteroaliphatic, optionally substituted aryl, optionally substituted heteroaryl or a suitable thiol protecting group; wherein each R^{A1c} is, independently, hydrogen, an optionally substituted aliphatic, optionally substituted heteroaliphatic, optionally substituted aryl, optionally substituted heteroaryl or a suitable amino protecting group, or two R^{A1c} groups are joined together to form a heterocyclic or heteroaryl group; and wherein each R^{A1d} is, independently, an optionally substituted aliphatic, optionally substituted heteroaliphatic, optionally substituted aryl, or an optionally substituted heteroaryl group; and

x is an integer between 0-4, inclusive.

In certain embodiments, each instance of R^{A1} is, independently, hydrogen, halogen, optionally substituted aliphatic, optionally substituted heteroaliphatic, optionally substituted aryl, optionally substituted heteroaryl, —CN, —NO₂, —NC, —OR^{A1a}. In certain embodiments, each instance of R^{A1} is, independently, hydrogen, halogen, optionally substituted C₁₋₆ alkyl, —NO₂, —CF₃, or —OR^{A1a}. In certain embodiments, each instance of R^{A1} is, independently, hydrogen, —CH₃, -tBu, —CN, —NO₂, —CF₃, or —OCH₃. In certain embodiments, each instance of R^{A1} is hydrogen.

In certain embodiments, R³ and R⁴ are joined to form an optionally substituted aryl ring to provide a palladium (II) complex of the formula (I-c):

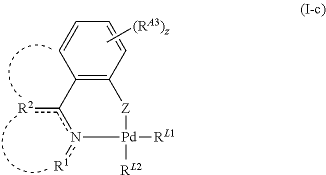

wherein

Pd, [symbol] ,

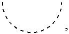

R¹, R², R^{L1}, R^{L2}, and Z are as defined above and herein;

each instance of R^{A3} is, independently, hydrogen, halogen, optionally substituted aliphatic, optionally substituted heteroaliphatic, optionally substituted aryl, optionally substituted heteroaryl, —CN, —NO₂, —NC, —OR^{A3a}, —SR^{A3b}, —N(R^{A3c})₂, —C(═O)R^{A3d}, —C(═O)OR^{A3a}, —C(═O)N(R^{A3c})₂, —C(═NR^{A3c})R^{A3d}, —C(═NR^{A3c})OR^{A3a}, —C(═NR^{A3c})N(R^{A3c})₂, —S(O)₂R^{A3d}, —S(O)R^{A3d}, or two R^{A3} groups adjacent to each other are joined to form a 5- to 6-membered aryl, heteroaryl, heterocyclic or carbocyclic ring, wherein R^{A3a} is hydrogen, an optionally substituted aliphatic, optionally substituted heteroaliphatic, optionally substituted aryl, optionally substituted heteroaryl or a suitable hydroxyl protecting group; wherein R^{A3b} is hydrogen, an optionally substituted aliphatic, optionally substituted heteroaliphatic, optionally substituted aryl, optionally substituted heteroaryl or a suitable thiol protecting group; wherein each R^{A3c} is, independently, hydrogen, an optionally substituted aliphatic, optionally substituted heteroaliphatic, optionally substituted aryl, optionally substituted heteroaryl or a suitable amino protecting group, or two R^{A3c} groups are joined together to form a heterocyclic or heteroaryl group; and wherein each R^{A3d} is, independently, an optionally substituted aliphatic, optionally substituted heteroaliphatic, optionally substituted aryl, or an optionally substituted heteroaryl group; and

z is an integer between 0-3, inclusive.

In certain embodiments, each instance of R^{A3} is, independently, hydrogen, halogen, optionally substituted aliphatic, optionally substituted heteroaliphatic, optionally substituted aryl, optionally substituted heteroaryl, —CN, —NO₂, —NC, —OR^{A3a}. In certain embodiments, each instance of R^{A3} is, independently, hydrogen, halogen, optionally substituted C₁₋₆ alkyl, —NO₂, —CF₃, or —OR^{A3a}. In certain embodiments, each instance of R^{A3} is, independently, hydrogen, —CH₃, -tBu, —CN, —NO₂, —CF₃, or —OCH₃. In certain embodiments, each instance of R^{A3} is hydrogen.

In certain embodiments, R¹ and R² are joined to form an optionally substituted 6-membered pyridinyl ring and R³ and R⁴ are joined to form an optionally substituted aryl ring to provide a palladium (II) complex of the formula (I-d):

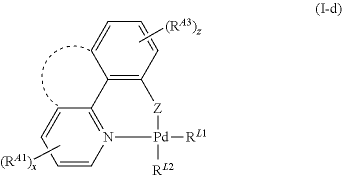

wherein Pd, [symbol] ,

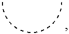

R^{A1}, R^{A3}, R^{L1}, R^{L2}, x, z, and Z are as defined above and herein.

In certain embodiments, R¹ and R² are joined to form an optionally substituted 6-membered pyridinyl ring and R² and R³ are joined to form an optionally substituted 6-membered aryl ring, to provide a palladium (II) catalyst of the formula (I-e):

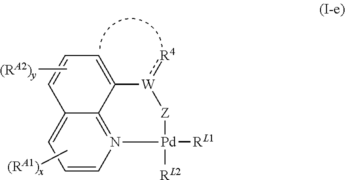

wherein

Pd, [symbol] ,

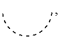

W, R^{A1}, R^{L1}, R^{L2}, R⁴, x, and Z are as defined above and herein;

each instance of R^{A2} is, independently, hydrogen, halogen, optionally substituted aliphatic, optionally substituted heteroaliphatic, optionally substituted aryl, optionally substituted heteroaryl, —CN, —NO₂, —NC, —OR^{A2a}, —SR^{A2b}, —N(R^{A2c})₂, —C(═O)R^{A2d}, —C(═O)OR^{A2a}, —C(═O)N(R^{A2c})₂, —C(═NR^{A2c})R^{A2d}, —C(═NR^{A2c})OR^{A2a}, —C(═NR^{A2c})N(R^{A2c})₂, —S(O)₂R^{A2d}, —S(O)R^{A2d}, or two R^{A2} groups adjacent to each other are joined to form a 5- to 6-membered aryl, heteroaryl, heterocyclic or carbocyclic ring, wherein R^{A2a} is hydrogen, an optionally substituted aliphatic, optionally substituted heteroaliphatic, optionally substituted aryl, optionally substituted heteroaryl or a suitable hydroxyl protecting group; wherein R^{A2b} is hydrogen, an optionally substituted aliphatic, optionally substituted heteroaliphatic, optionally substituted aryl, optionally substituted heteroaryl or a suitable thiol protecting group; wherein each R^{A2c} is, independently, hydrogen, an optionally substituted aliphatic, optionally substituted heteroaliphatic, optionally substituted aryl, optionally substituted heteroaryl or a suitable amino protecting group, or two R^{A2c} groups are joined together to form a heterocyclic or heteroaryl group; and wherein each R^{A2d} is, independently, an optionally substituted aliphatic, optionally substituted heteroaliphatic, optionally substituted aryl, or an optionally substituted heteroaryl group; and

y is an integer between 0-2, inclusive.

In certain embodiments, each instance of R^{A2} is, independently, hydrogen, halogen, optionally substituted aliphatic, optionally substituted heteroaliphatic, optionally substituted aryl, optionally substituted heteroaryl, —CN, —NO₂, —NC, —OR^{A2a}. In certain embodiments, each instance of R^{A2} is, independently, hydrogen, halogen, optionally substituted C₁₋₆ alkyl, —NO₂, —CF₃, or —OR^{A2a}. In certain embodiments, each instance of R^{A2} is, independently, hydrogen, —CH₃, -tBu, —CN, —NO₂, —CF₃, or —OCH₃. In certain embodiments, each instance of R^{A2} is hydrogen.

In certain embodiments, R² and R³ are joined to form an optionally substituted 6-membered aryl ring to provide a palladium (II) catalyst of the formula (I-f):

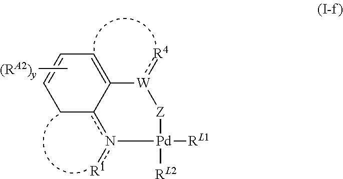

wherein Pd, [symbol] ,

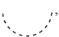

W, R^{A2}, R¹, R⁴, R^{L1}, R^{L2}, y and Z are as defined above and herein.

In certain embodiments, R¹ and R² are joined to form an optionally substituted pyridinyl ring, R² and R³ are joined to form an optionally substituted 6-membered aryl ring and R³ and R⁴ are joined to form an optionally substituted 6-membered aryl ring to form the bidentate palladium (II) complex of the formula (I-g):

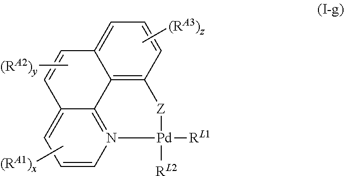

wherein Pd, R^{L1}, R^{L2}, Z, R^{A1}, R^{A2}, R^{A3}, x, y and z are as defined above and herein.

In certain embodiments, wherein R² and R³ are not joined to form an optionally substituted 5- to 6-membered ring, the palladium (II) complex is of the formula (I-h):

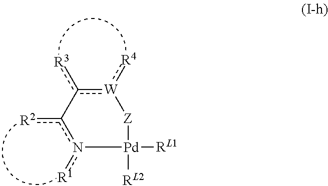

wherein Pd, [symbol] ,

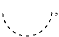

W, Z, R¹, R², R³, R⁴, R^{L1} and R^{L2} are as defined above and herein; and

R¹, R², R³ and R⁴ are, independently, an optionally substituted aliphatic, optionally substituted heteroaliphatic, optionally substituted aryl, optionally substituted heteroaryl group,

R¹ and R² are optionally joined to form an optionally substituted 5- to 7-membered heteroaryl, aryl, heterocyclic or carbocyclic ring; and

R³ and R⁴ are optionally joined to form an optionally substituted 5- to 7-membered heteroaryl, aryl, heterocyclic or carbocyclic ring.

In certain embodiments, wherein R² and R³ are not joined to form a cyclic structure, the palladium (II) complex is of the formula (I-i):

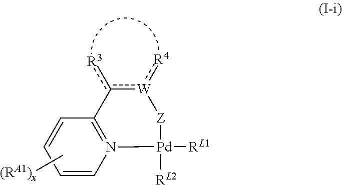

wherein Pd, [symbol] ,

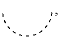

W, R³, R⁴, R^{L1}, R^{L2}, R^{A1} and x are as defined above and herein.

In certain embodiments, wherein R² and R³ are not joined to form a cyclic structure, the palladium (II) complex is of the formula (I-j):

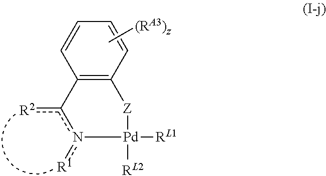

wherein Pd, [symbol] ,

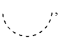

R¹, R², R^{L1}, R^{L2}, R^{A3}, Z, and z are as defined above and herein.

In certain embodiments, wherein R² and R³ are not joined to form a cyclic structure, the palladium (II) complex is of the formula (I-k):

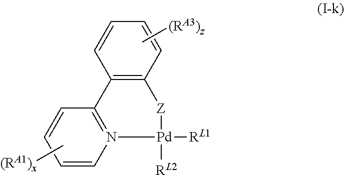

wherein Pd, R^{L1}, R^{L2}, R^{A1}, R^{A3}, Z, z and x are as defined above and herein.

In certain embodiments, in any of the above formulae Z is a bond. In other embodiments, Z is

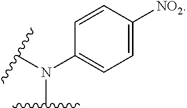

In other embodiments, Z is

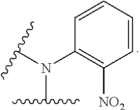

In certain embodiments, wherein R² and R³ are not joined to form a cyclic structure and Z is a bond, the palladium (II) complex is of the formula (I-l):

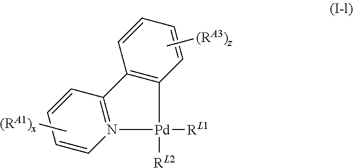

wherein R^{L1}, R^{L2}, R^{A1}, R^{A3}, z and x are as defined above and herein.

In certain embodiments, the palladium (II) complex is of the formula (I-k):

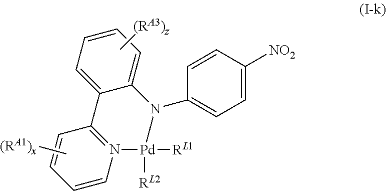

wherein R^{L1}, R^{L2}, R^{A1}, R^{A3}, z, and x are as defined above and herein.

In certain embodiments, the palladium (II) complex is of the formula (I-l′):

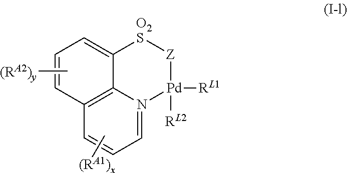

wherein Pd, R^{L1}, R^{L2}, R^{A1}, R^{A2}, x, y, and Z are as defined above and herein.

In certain embodiments, the palladium (II) complex is of the formula (I-m′):

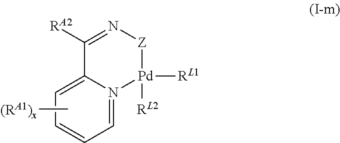

wherein Pd, R^{L1}, R^{L2}, R^{A1}, R^{A2}, x, and Z are as defined above and herein.

In certain embodiments, the palladium (II) complex is of the formula (I-n′):

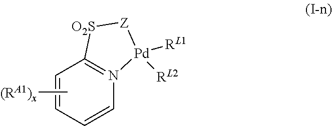

wherein Pd, R^{L1}, R^{L2}, R^{A1}, x, and Z are as defined above and herein.

Palladium (II) Complexes with Tridentate Ligand

In certain embodiments, Z is joined via a linker group -L- to the group R^{L1} to form a 5- to 7-membered palladacycle.

In certain embodiments, the palladium (II) catalyst comprises a tridentate ligand. In certain embodiments, the palladium (II) catalyst of the formula (I-a′):

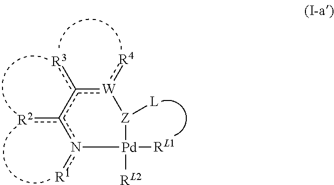

wherein

Pd, [symbol] ,

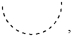

W, R^{L1}, R^{L2}, R¹, R², R³, and R⁴ are as defined above and herein;

Z is —N— joined via a linker group -L- to the group R^{L1} to form a 5- to 7-membered palladacycle, wherein -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} is an optionally substituted aryl, optionally substituted heteroaryl, or an —N(R^{c})₂ group wherein two R^{c} groups are joined to form an optionally substituted heterocyclic or heteroaryl ring; and

the curved solid line

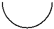

represents joining of the 5- to 7-membered palladacycle.

In certain embodiments, R¹ and R² are joined to form an optionally substituted 6-membered pyridinyl ring to provide a palladium (II) complex of the formula (I-b′):

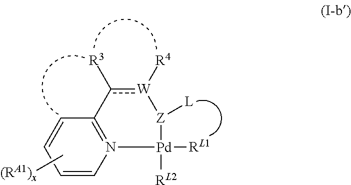

wherein

Pd, [symbol] ,

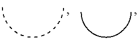

W, L, R^{L1}, R^{L2}, Z, R³ and R⁴ are as defined above and herein;

each instance of R^{A1} is, independently, hydrogen, halogen, optionally substituted aliphatic, optionally substituted heteroaliphatic, optionally substituted aryl, optionally substituted heteroaryl, —CN, —NO₂, —NC, —OR^{A1a}, —SR^{A1b}, —N(R^{A1c})₂, —C(═O)R^{A1d}, —C(═O)OR^{A1a}, —C(═O)N(R^{A1c})₂, —C(═NR^{A1c})R^{A1d}, —C(═NR^{A1c})OR^{A1a}, —C(═NR^{A1c})N(R^{A1c})₂, —S(O)₂R^{A1d}, —S(O)R^{A1d}, or two R^{A1} groups adjacent to each other are joined to form a 5- to 6-membered aryl, heteroaryl, heterocyclic or carbocyclic ring, wherein R^{A1a} is hydrogen, an optionally substituted aliphatic, optionally substituted heteroaliphatic, optionally substituted aryl, optionally substituted heteroaryl or a suitable hydroxyl protecting group; wherein R^{A1b} is hydrogen, an optionally substituted aliphatic, optionally substituted heteroaliphatic, optionally substituted aryl, optionally substituted heteroaryl or a suitable thiol protecting group; wherein each R^{A1c} is, independently, hydrogen, an optionally substituted aliphatic, optionally substituted heteroaliphatic, optionally substituted aryl, optionally substituted heteroaryl or a suitable amino protecting group, or two R^{A1c} groups are joined together to form a heterocyclic or heteroaryl group; and wherein each R^{A1d} is, independently, an optionally substituted aliphatic, optionally substituted heteroaliphatic, optionally substituted aryl, or an optionally substituted heteroaryl group; and

x is an integer between 0-4, inclusive.

In certain embodiments, each instance of R^{A1} is, independently, hydrogen, halogen, optionally substituted aliphatic, optionally substituted heteroaliphatic, optionally substituted aryl, optionally substituted heteroaryl, —CN, —NO₂, —NC, —OR^{A1a}. In certain embodiments, each instance of R^{A1} is, independently, hydrogen, halogen, optionally substituted C₁₋₆ alkyl, —NO₂, —CF₃, or —OR^{A1a}. In certain embodiments, each instance of R^{A1} is, independently, hydrogen, —CH₃, -tBu, —CN, —NO₂, —CF₃, or —OCH₃. In certain embodiments, each instance of R^{A1} is hydrogen.

In certain embodiments, R³ and R⁴ joined to form an optionally substituted aryl ring to provide a palladium (II) complex of the formula (I-c′):

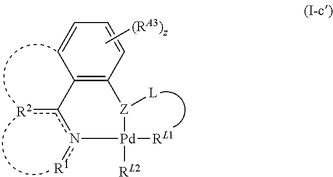

wherein

Pd, [symbol] ,

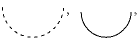

L, R¹, R², R^{L1}, R^{L2}, z, and Z are as defined above and herein;

each instance of R^{A3} is, independently, hydrogen, halogen, optionally substituted aliphatic, optionally substituted heteroaliphatic, optionally substituted aryl, optionally substituted heteroaryl, —CN, —NO₂, —NC, —OR^{A3a}, —SR^{A3b}, —N(R^{A3c})₂, —C(═O)R^{A3d}, —C(═O)OR^{A3a}, —C(═O)N(R^{A3c})₂, —C(═NR^{A3c})R^{A3d}, —C(═NR^{A3c})OR^{A3a}, —C(═NR^{A3c})N(R^{A3c})₂, —S(O)₂R^{A3d}, —S(O)R^{A3d}, or two R^{A3} groups adjacent to each other are joined to form a 5- to 6-membered aryl, heteroaryl, heterocyclic or carbocyclic ring, wherein R^{A3a} is hydrogen, an optionally substituted aliphatic, optionally substituted heteroaliphatic, optionally substituted aryl, optionally substituted heteroaryl or a suitable hydroxyl protecting group; wherein R^{A3b} is hydrogen, an optionally substituted aliphatic, optionally substituted heteroaliphatic, optionally substituted aryl, optionally substituted heteroaryl or a suitable thiol protecting group; wherein each R^{A3c} is, independently, hydrogen, an optionally substituted aliphatic, optionally substituted heteroaliphatic, optionally substituted aryl, optionally substituted heteroaryl or a suitable amino protecting group, or two R^{A3c} groups are joined together to form a heterocyclic or heteroaryl group; and wherein each R^{A3d} is, independently, an optionally substituted aliphatic, optionally substituted heteroaliphatic, optionally substituted aryl, or an optionally substituted heteroaryl group; and

z is an integer between 0-3, inclusive.

In certain embodiments, each instance of R^{A3} is, independently, hydrogen, halogen, optionally substituted aliphatic, optionally substituted heteroaliphatic, optionally substituted aryl, optionally substituted heteroaryl, —CN, —NO₂, —NC, —OR^{A3a}. In certain embodiments, each instance of R^{A3} is, independently, hydrogen, halogen, optionally substituted C₁₋₆ alkyl, —NO₂, —CF₃, or —OR^{A3a}. In certain embodiments, each instance of R^{A3} is, independently, hydrogen, —CH₃, -tBu, —CN, —NO₂, —CF₃, or —OCH₃. In certain embodiments, each instance of R^{A3} is hydrogen.

In certain embodiments, R¹ and R² are joined to form an optionally substituted 6-membered pyridinyl ring and R³ and R⁴ are joined to form an optionally substituted aryl ring to provide a palladium (II) complex of the formula (I-d′):

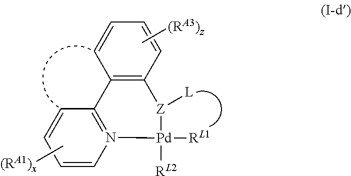

wherein Pd, [symbol] ,

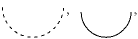

L, R^{A1}, R^{A3}, R^{L1}, R^{L2}, x, z, and Z are as defined above and herein.

In certain embodiments, R¹ and R² are joined to form an optionally substituted 6-membered pyridinyl ring and R² and R³ are joined to form an optionally substituted 6-membered aryl ring, to provide a palladium (II) catalyst of the formula (I-e′):

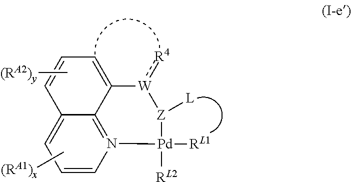

wherein Pd, [symbol] ,

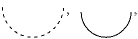

L, W, R^{A1}, R^{L1}, R^{L2}, R⁴, x, and Z are as defined above and herein;

each instance of R^{A2} is, independently, hydrogen, halogen, optionally substituted aliphatic, optionally substituted heteroaliphatic, optionally substituted aryl, optionally substituted heteroaryl, —CN, —NO₂, —NC, —OR^{A2a}, —SR^{A2b}, —N(R^{A2c})₂, —C(═O)R^{A2d}, —C(═O)OR^{A2a}, —C(═O)N(R^{A2c})₂, —C(═NR^{A2c})R^{A2d}, —C(═NR^{A2c})OR^{A2a}, —C(═NR^{A2c})N(R^{A2c})₂, —S(O)₂R^{A2d}, —S(O)R^{A2d}, or two R^{A2} groups adjacent to each other are joined to form a 5- to 6-membered aryl, heteroaryl, heterocyclic or carbocyclic ring, wherein R^{A2a} is hydrogen, an optionally substituted aliphatic, optionally substituted heteroaliphatic, optionally substituted aryl, optionally substituted heteroaryl or a suitable hydroxyl protecting group; wherein R^{A2b} is hydrogen, an optionally substituted aliphatic, optionally substituted heteroaliphatic, optionally substituted aryl, optionally substituted heteroaryl or a suitable thiol protecting group; wherein each R^{A2c} is, independently, hydrogen, an optionally substituted aliphatic, optionally substituted heteroaliphatic, optionally substituted aryl, optionally substituted heteroaryl or a suitable amino protecting group, or two R^{A2c} groups are joined together to form a heterocyclic or heteroaryl group; and wherein each R^{A2d} is, independently, an optionally substituted aliphatic, optionally substituted heteroaliphatic, optionally substituted aryl, or an optionally substituted heteroaryl group; and

y is an integer between 0-2, inclusive.

In certain embodiments, each instance of R^{A2} is, independently, hydrogen, halogen, optionally substituted aliphatic, optionally substituted heteroaliphatic, optionally substituted aryl, optionally substituted heteroaryl, —CN, —NO₂, —NC, —OR^{A2a}. In certain embodiments, each instance of R^{A2} is, independently, hydrogen, halogen, optionally substituted C₁₋₆ alkyl, —NO₂, —CF₃, or —OR^{A2a}. In certain embodiments, each instance of R^{A2} is, independently, hydrogen, —CH₃, -tBu, —CN, —NO₂, —CF₃, or —OCH₃. In certain embodiments, each instance of R^{A2} is hydrogen.

In certain embodiments, R² and R³ joined to form an optionally substituted 6-membered aryl ring to provide a palladium (II) catalyst of the formula (I-f′):

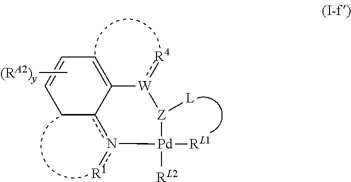

wherein Pd, [symbol] ,

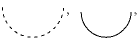

L, W, R^{A2}, R¹, R⁴, R^{L1}, R^{L2}, y and Z are as defined above and herein.

In certain embodiments, R¹ and R² are joined to form an optionally substituted pyridinyl ring, R² and R³ are joined to form an optionally substituted 6-membered aryl ring and R³ and R⁴ are joined to form an optionally substituted 6-membered aryl ring to form the palladium (II) complex of the formula (I-g′):

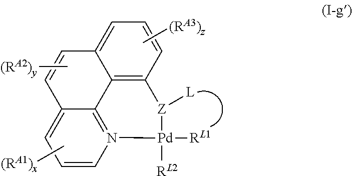

wherein

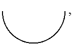

L, R^{L1}, R^{L2}, Z, R^{A1}, R^{A2}, R^{A3}, x, y and z are as defined above and herein.

In certain embodiments, wherein R² and R³ are not joined to form an optionally substituted 5- to 6-membered ring, the palladium (II) complex is of the formula (I-h′):

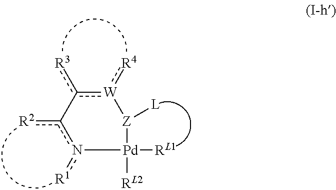

wherein Pd, [symbol] ,

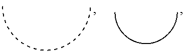

L, W, Z, R¹, R², R³, R⁴, R^{L1} and R¹² are as defined above and herein; and

R¹, R², R³ and R⁴ are, independently, an optionally substituted aliphatic, optionally substituted heteroaliphatic, optionally substituted aryl, optionally substituted heteroaryl group,

R¹ and R² are optionally joined to form an optionally substituted 5- to 7-membered heteroaryl, aryl, heterocyclic or carbocyclic ring;

and

R³ and R⁴ are optionally joined to form an optionally substituted 5- to 7-membered heteroaryl, aryl, heterocyclic or carbocyclic ring.

In certain embodiments, wherein R² and R³ are not joined to form a cyclic structure, the palladium (II) complex is of the formula (I-i′):

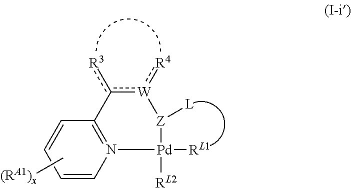

wherein Pd, [symbol] ,

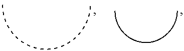

L, W, R³, R⁴, R^{L1}, R^{L2}, R^{A1}, and x are as defined above and herein.

In certain embodiments, wherein R² and R³ are not joined to form a cyclic structure, the palladium (II) complex is of the formula (I-j′):

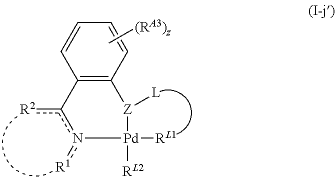

wherein Pd, [symbol] ,

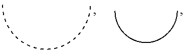

L, R¹, R², R^{L1}, R^{L2}, R^{A3}, and z are as defined above and herein.

In certain embodiments, wherein R² and R³ are not joined to form a cyclic structure, the palladium (II) complex is of the formula (I-k′):

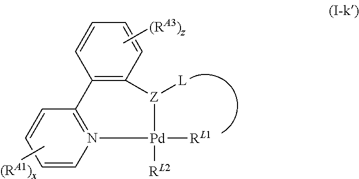

wherein Pd,

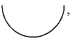

L, R^{L1}, R^{L2}, R^{A1}, R^{A3}, Z, z and x are as defined above and herein.

Groups R^{L1} and R^{L2}

As defined generally above, R^{L1} and R^{L2} are, independently, halogen, optionally substituted aliphatic, optionally substituted heteroaliphatic, optionally substituted aryl, optionally substituted heteroaryl, —OR^{a}, —SR^{b}, —N(R^{c})₃, —N(R^{c})₂, or —P(R^{x})₃,

wherein each instance of R^{a} is, independently, hydrogen, an optionally substituted aliphatic, optionally substituted heteroaliphatic, optionally substituted aryl, optionally substituted heteroaryl, —C(═O)R^{a1}, —C(═O)OR^{a2}, —C(═O)N(R^{a3})₂, —C(═NR^{a3})R^{a3}, —C(═NR^{a3})OR^{a1}, —C(═NR^{a3})N(R^{a3})₂, —S(O)₂R^{a1}, —S(O)R^{a1}, or a suitable hydroxyl protecting group, wherein R^{a1} is an optionally substituted aliphatic, optionally substituted heteroaliphatic, optionally substituted aryl or optionally substituted heteroaryl group; wherein R^{a2} is an optionally substituted aliphatic, optionally substituted heteroaliphatic, optionally substituted aryl, optionally substituted heteroaryl group, or a suitable hydroxyl protecting group; wherein R^{a3} is an optionally substituted aliphatic, optionally substituted heteroaliphatic, optionally substituted aryl, optionally substituted heteroaryl group, or a suitable amino protecting group, or two R^{a3} groups are joined to form an optionally substituted heterocyclic or heteroaryl ring;

wherein each instance of R^{b} is, independently, an optionally substituted aliphatic, heteroaliphatic, aryl, heteroaryl, —C(═O)R^{b1}, —C(═O)OR^{b2}, —C(═O)N(R^{b3})₂, —C(═NR^{b3})R^{b3}, —C(═NR^{b3})OR^{b1}, —C(═NR^{a3})N(R^{b3})₂, or a suitable thiol protecting group, wherein R^{b1} is an optionally substituted aliphatic, optionally substituted heteroaliphatic, optionally substituted aryl or optionally substituted heteroaryl group; wherein R^{b2} is an optionally substituted aliphatic, optionally substituted heteroaliphatic, optionally substituted aryl, optionally substituted heteroaryl group, or a suitable hydroxyl protecting group; wherein R^{b3} is an optionally substituted aliphatic, optionally substituted heteroaliphatic, optionally substituted aryl, optionally substituted heteroaryl group, or a suitable amino protecting group, or two R^{b3} groups are joined to form an optionally substituted heterocyclic or heteroaryl ring;

wherein each instance of R^{c} is, independently, hydrogen, an optionally substituted aliphatic, heteroaliphatic, aryl, heteroaryl, —C(═O)R^{c1}, —C(═O)OR^{c2}, —C(═O)N(R^{c3})₂, —C(═NR^{c3})R^{c3}, —C(═NR^{c3})OR^{c1}, —C(═NR^{c3})N(R^{c3})₂, —S(O)₂R^{c1}, —S(O)R^{c1}, or a suitable amino protecting group, or two R^{c} groups are joined to form an optionally substituted 5- to 6-membered heterocyclic or heteroaryl ring or the group ≡C(R^{c1}), wherein R^{c1} is an optionally substituted aliphatic, optionally substituted heteroaliphatic, optionally substituted aryl or optionally substituted heteroaryl group; wherein R^{c2} is an optionally substituted aliphatic, optionally substituted heteroaliphatic, optionally substituted aryl, optionally substituted heteroaryl group, or a suitable hydroxyl protecting group; wherein R^{c3} is an optionally substituted aliphatic, optionally substituted heteroaliphatic, optionally substituted aryl, optionally substituted heteroaryl group, or a suitable amino protecting group, or two R^{c3} groups are joined to form an optionally substituted heterocyclic or heteroaryl ring; and

wherein each instance of R^{x} is, independently, hydrogen, an optionally substituted aliphatic, optionally substituted alkoxy, optionally substituted heteroaliphatic, optionally substituted aryloxy, optionally substituted heteroaryloxy, optionally substituted aryl, or optionally substituted heteroaryl group.

In certain embodiments, at least one of R^{L1} and R^{L2} is selected from halogen, —OR^{a}, —SR^{b}, —N(R^{c})₃, —N(R^{c})₂, or —P(R^{x})₃. In certain embodiments, both R^{L1} and R^{L2} are, independently, selected from halogen, —OR^{a}, —SR^{b}, —N(R^{c})₃, —N(R^{c})₂, or —P(R^{x})₃.

In certain embodiments, R^{L1} is halogen, —OR^{a}, —SR^{b}, or —N(R^{c})₂, and R^{L2} is —N(R^{c})₂. In certain embodiments, R^{L1} is halogen, —OR^{a} or —N(R^{c})₂, and R^{L2} is —N(R^{c})₂. In certain embodiments, R^{L1} is halogen or —OR^{a}, and R^{L2} is —N(R^{c})₂. In certain embodiments, R^{L1} is and R^{L2} is —N(R^{c})₂. In certain embodiments, R^{L1} halogen and R^{L2} is —N(R^{c})₂. In certain embodiments, R^{L1} is —OR^{a} and R^{L2} is —N(R^{c})₂. In certain embodiments, both R^{L1} and R^{L2} are independently —N(R^{c})₂.

In certain embodiments, R^{L1} is halogen. In certain embodiments, R^{L1} is —Cl. In certain embodiments, R^{L1} is —Br. In certain embodiments, R^{L1} is —I. In certain embodiments, R^{L1} is —F.

In certain embodiments, R^{L1} is —OR^{a}.

In certain embodiments, R^{L1} is —OC(═O)R^{a1} wherein R^{a1} is an optionally substituted aliphatic, optionally substituted heteroaliphatic, optionally substituted aryl or optionally substituted heteroaryl group. In certain embodiments, R^{L1} is —OC(═O)R^{a1} wherein R^{a1} is an optionally substituted aliphatic group. In certain embodiments, R^{L1} is —OC(═O)R^{a1} wherein R^{a1} is an optionally substituted C₁₋₆ alkyl group. In certain embodiments, R^{L1} is —OC(═O)R^{a1} wherein R^{a1} is an optionally substituted C₁₋₄ alkyl group. In certain embodiments, R^{L1} is —OC(═O)R^{a1} wherein R^{a1} is an optionally substituted C₁₋₂ alkyl group. In certain embodiments, R^{L1} is —OC(═O)CH₃.

In certain embodiments, R^{L1} is —P(R^{X})₃.

In certain embodiments, R^{L2} is —N(R^{c})₂.

In certain embodiments, R^{L2} is —N(R^{c})₂ wherein two R^{c} groups are joined to form the group ≡C(R^{c1}), wherein R^{c1} is an optionally substituted aliphatic, optionally substituted heteroaliphatic, optionally substituted aryl or optionally substituted heteroaryl group. In certain embodiments, R^{L2} is —N(R^{c})₂ wherein two R^{c} groups are joined to form the group ≡C(R^{c1}), wherein R^{c1} is an optionally substituted aliphatic group. In certain embodiments, R^{L2} is —N(R^{c})₂ wherein two R^{c} groups are joined to form the group ≡C(R^{c1}), wherein R^{c1} is an optionally substituted C₁₋₆ alkyl group. In certain embodiments, R^{L2} is —N(R^{c})₂ wherein two R^{c} groups are joined to form the group ≡C(CH₃) or ≡C(CH₂Ph).

In certain embodiments, R^{L2} is —N(R^{c})₂ wherein two R^{c} groups are joined to form an optionally substituted heterocyclic or heteroaryl ring.

In certain embodiments, R^{L2} is —N(R^{c})₂ wherein two R^{c} groups are joined to form an optionally substituted 5- to 6-membered heterocyclic or heteroaryl ring.

In certain embodiments, R^{L2} is —N(R^{c})₂ wherein two R^{c} groups are joined to form an optionally substituted 5-membered heterocyclic ring. Exemplary 5-membered heterocyclic rings include, but are not limited to, an optionally substituted pyrrolidinyl ring.

In certain embodiments, R^{L2} is —N(R^{c})₂ wherein two R^{c} groups are joined to form an optionally substituted 5-membered heteroaryl ring. Exemplary 5-membered heteroaryl rings include, but are not limited to, an optionally substituted pyrrolyl, optionally substituted pyrazolyl, optionally substituted imidazolyl, optionally substituted triazolyl or optionally substituted tetrazolyl, optionally substituted thiazolyl, optionally substituted isothazolyl, optionally substituted thiadiazolyl, optionally substituted oxazolyl, optionally substituted isooxazolyl, optionally substituted oxadiaziolyl or optionally substituted oxadiaziolyl ring.

In certain embodiments, R^{L2} is —N(R^{c})₂ wherein two R^{c} groups are joined to form an optionally substituted 6-membered heterocyclic ring. Exemplary 6-membered heterocyclic rings include, but are not limited to, optionally substituted piperidinyl, optionally substituted piperazinyl or optionally substituted morpholinyl ring.

In certain embodiments, R^{L2} is —N(R^{c})₂ wherein two R^{c} groups are joined to form an optionally substituted 6-membered heteroaryl ring. Exemplary 6-membered heteroaryl rings include, but are not limited to, optionally substituted pyridinyl, optionally substituted pyrimidinyl, optionally substituted pyrazinyl, optionally substituted pyridazinyl, optionally substituted triazinyl or optionally substituted tetrazinyl ring.

In certain embodiments, R^{L2} is an optionally substituted pyridinyl ring.

In certain embodiments, R^{L1} is —N(R^{c})₂.

In certain embodiments, R^{L1} is —N(R^{c})₂ wherein two R^{c} groups are joined to form the group ≡C(R^{c1}), wherein R^{c1} is an optionally substituted aliphatic, optionally substituted heteroaliphatic, optionally substituted aryl or optionally substituted heteroaryl group. In certain embodiments, R^{L1} is —N(R^{c})₂ wherein two R^{c} groups are joined to form the group ≡C(R^{c1}), wherein R^{c1} is an optionally substituted aliphatic group. In certain embodiments, R^{L1} is —N(R^{c})₂ wherein two R^{c} groups are joined to form the group ≡C(R^{c1}), wherein R^{c1} is an optionally substituted C₁₋₆ alkyl group. In certain embodiments, R^{L1} is —N(R^{c})₂ wherein two R^{c} groups are joined to form the group ≡C(CH₃) or ≡C(CH₂Ph).

In certain embodiments, R^{L1} is —N(R^{c})₂ wherein two R^{c} groups are joined to form an optionally substituted 5- to 6-membered heterocyclic or heteroaryl ring.

In certain embodiments, R^{L1} is —N(R^{c})₂ wherein two R^{c} groups are joined to form an optionally substituted 5-membered heterocyclic ring. Exemplary 5-membered heterocyclic rings are provided above and herein.

In certain embodiments, R^{L1} is —N(R^{c})₂ wherein two R^{c} groups are joined to form an optionally substituted 5-membered heteroaryl ring. Exemplary 5-membered heteroaryl rings are provided above and herein.

In certain embodiments, R^{L1} is —N(R^{c})₂ wherein two R^{c} groups are joined to form an optionally substituted 6-membered heterocyclic ring. Exemplary 6-membered heterocyclic rings are provided above and herein.

In certain embodiments, R^{L1} is —N(R^{c})₂ wherein two R^{c} groups are joined to form an optionally substituted 6-membered heteroaryl ring. Exemplary 6-membered heteroaryl rings are provided above and herein.

In certain embodiments, R^{L1} is an optionally substituted pyridinyl ring.

Optionally substituted pyridinyl rings include, but are not limited to, rings of the formula:

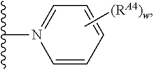

wherein each instance of R^{A4} is, independently, hydrogen, halogen, optionally substituted aliphatic, optionally substituted heteroaliphatic, optionally substituted aryl, optionally substituted heteroaryl, —CN, —NO₂, —NC, —OR^{A4a}, —SR^{A4b}, —N(R^{A4c})₂, —C(═O)R^{A4d}, —C(═O)OR^{A4a}, —C(═O)N(R^{A4c})₂, —C(═NR^{A4c})R^{A4d}, —C(═NR^{A4c})OR^{A4a}, —C(═NR^{A4c})N(R^{A4c})₂, —S(O)₂R^{A4d}, —S(O)R^{A4d}, or two R^{A4} groups adjacent to each other are joined to form a 5- to 6-membered aryl, heteroaryl, heterocyclic or carbocyclic ring, wherein R^{A4a} is hydrogen, an optionally substituted aliphatic, optionally substituted heteroaliphatic, optionally substituted aryl, optionally substituted heteroaryl or a suitable hydroxyl protecting group; wherein R^{A4b} is hydrogen, an optionally substituted aliphatic, optionally substituted heteroaliphatic, optionally substituted aryl, optionally substituted heteroaryl or a suitable thiol protecting group; wherein each R^{A4c} is, independently, hydrogen, an optionally substituted aliphatic, optionally substituted heteroaliphatic, optionally substituted aryl, optionally substituted heteroaryl or a suitable amino protecting group, or two R^{A4c} groups are joined together to form a heterocyclic or heteroaryl group; and wherein each R^{A4d} is, independently, an optionally substituted aliphatic, optionally substituted heteroaliphatic, optionally substituted aryl, or an optionally substituted heteroaryl group, and

w is an integer between 0 to 5, inclusive.

In certain embodiments, the optionally substituted pyridinyl ring is of the formulae:

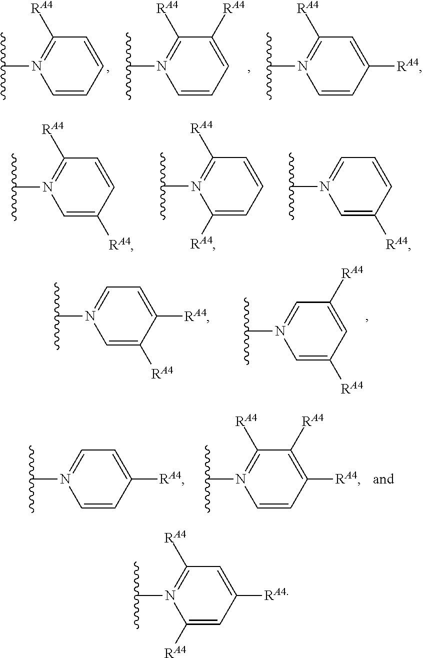

In certain embodiments, the optionally substituted pyridinyl ring is:

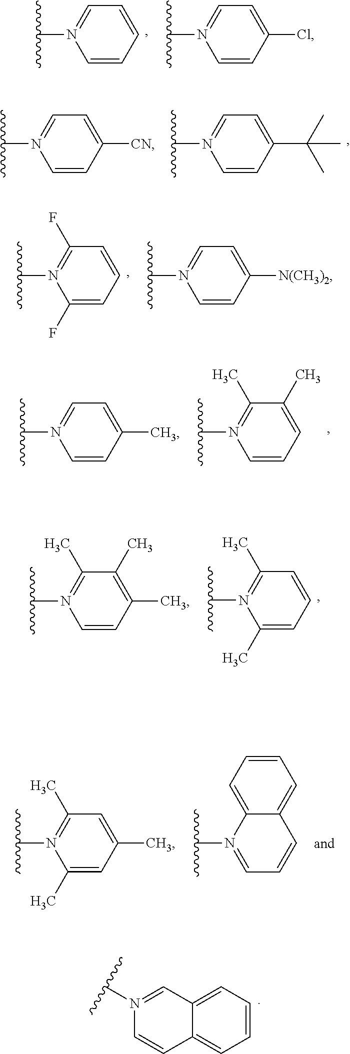

In certain embodiments, R^{L2} is —P(R^{X})₃. In certain embodiments, R^{X} is optionally substituted aliphatic. In certain embodiments, R^{X} is optionally substituted aryl. In certain embodiments, R^{X} is optionally substituted alkoxy. In certain embodiments, R^{X} is optionally substituted aryloxy. In certain embodiments, R^{L2} is —P(Me)₃. In certain embodiments, R^{L2} is —P(Et)₃. In certain embodiments, R^{L2} is —P(tert-Bu)₃. In certain embodiments, R^{L2} is —P(Cy)₃. In certain embodiments, R^{L2} is —P(Ph)₃. In certain embodiments, R^{L2} is —PMe(Ph)₂. In certain embodiments, R^{L2} is —PF₃. In certain embodiments, R^{L2} is —P(OMe)₃. In certain embodiments, R^{L2} is —P(OEt)₃. In certain embodiments, R^{L2} is —P(OPh)₃.

Z, L, and R^{L1}

As generally defined above, in certain embodiments, Z is —N— joined via a linker group -L- to the group R^{L1} to form a 5- to 7-membered palladacycle, wherein -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} an optionally substituted aryl, optionally substituted heteroaryl, or an —N(R^{c})₂ group wherein two R^{c} groups are joined to form an optionally substituted membered heterocyclic or heteroaryl ring.

In certain embodiments, R^{L1} is —N(R^{c})₂ optionally joined to Z via a linker group -L- to form a 5- to 7-membered palladacycle, wherein two R^{c} groups are joined to form an optionally substituted membered heterocyclic or heteroaryl ring.

In certain embodiments, two R^{c} groups are joined to form an optionally substituted 5-membered heterocyclic ring. Exemplary 5-membered heterocyclic rings include, but are not limited to, an optionally substituted pyrrolidinyl ring.

In certain embodiments, two R^{c} groups are joined to form an optionally substituted 5-membered heteroaryl ring. Exemplary 5-membered heteroaryl rings include, but are not limited to, an optionally substituted pyrrolyl, optionally substituted pyrazolyl, optionally substituted imidazolyl, optionally substituted triazolyl or optionally substituted tetrazolyl, optionally substituted thiazolyl, optionally substituted isothazolyl, optionally substituted thiadiazolyl, optionally substituted oxazolyl, optionally substituted isooxazolyl, optionally substituted oxadiaziolyl or optionally substituted oxadiaziolyl ring.

In certain embodiments, two R^{c} groups are joined to form an optionally substituted 6-membered heterocyclic ring. Exemplary 6-membered heterocyclic rings include, but are not limited to, optionally substituted piperidinyl, optionally substituted piperazinyl or optionally substituted morpholinyl ring.

In certain embodiments, two R^{c} groups are joined to form an optionally substituted 6-membered heteroaryl ring. Exemplary 6-membered heteroaryl rings include, but are not limited to, optionally substituted pyridinyl, optionally substituted pyrimidinyl, optionally substituted pyrazinyl, optionally substituted pyridazinyl, optionally substituted triazinyl or optionally substituted tetrazinyl ring.

In certain embodiments, two R^{c} groups are joined to form an optionally substituted bicyclic heteroaryl ring. Exemplary bicyclic heteroaryl rings include, but are not limited to, optionally substituted quinolinyl and optionally substituted isoquinolinyl.

In certain embodiments, two R^{c} groups are joined to form an optionally substituted pyridinyl ring. In certain embodiments, two R^{c} groups are joined to form an optionally substituted quinolinyl ring.

For example, in certain embodiments, wherein two R^{c} groups are joined to form an optionally substituted pyridinyl ring, the group provided by Z, L and R^{L1} is of the formulae:

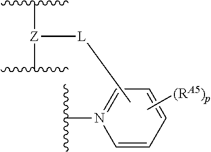

wherein:

Z is —N—;

L is -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)—, and

each instance of R^{A5} is, independently, hydrogen, halogen, optionally substituted aliphatic, optionally substituted heteroaliphatic, optionally substituted aryl, optionally substituted heteroaryl, —CN, —NO₂, —NC, —OR^{A5a}, —SR^{A5b}, —N(R^{A5c})₂, —C(═O)R^{A5d}, —C(═O)OR^{A5a}, —C(═O)N(R^{A5c})₂, —C(═NR^{A5c})R^{A5d}, —C(═NR^{A5c})OR^{A5a}, —C(═NR^{A5c})N(R^{A5c})₂, —S(O)₂R^{A5d}, —S(O)R^{A5d}, or two R^{A5} groups adjacent to each other are joined to form a 5- to 6-membered aryl, heteroaryl, heterocyclic or carbocyclic ring, wherein R^{A5a} is hydrogen, an optionally substituted aliphatic, optionally substituted heteroaliphatic, optionally substituted aryl, optionally substituted heteroaryl or a suitable hydroxyl protecting group; wherein R^{A5b} is hydrogen, an optionally substituted aliphatic, optionally substituted heteroaliphatic, optionally substituted aryl, optionally substituted heteroaryl or a suitable thiol protecting group; wherein each R^{A5c} is, independently, hydrogen, an optionally substituted aliphatic, optionally substituted heteroaliphatic, optionally substituted aryl, optionally substituted heteroaryl or a suitable amino protecting group, or two R^{A5c} groups are joined together to form a heterocyclic or heteroaryl group; and wherein each R^{A5d} is, independently, an optionally substituted aliphatic, optionally substituted heteroaliphatic, optionally substituted aryl, or an optionally substituted heteroaryl group, and

p is and integer between 0 to 5, inclusive.

In certain embodiments, wherein two R^{c} groups are joined to form an optionally substituted quinolinyl ring, the group provided by Z, L and R^{L1} is of the formulae:

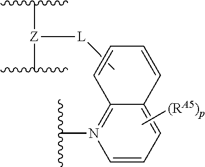

wherein:

Z is —N—;

L is -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)—, and

each instance of R^{A5} is, independently, hydrogen, halogen, optionally substituted aliphatic, optionally substituted heteroaliphatic, optionally substituted aryl, optionally substituted heteroaryl, —CN, —NO₂, —NC, —OR^{A5a}, —SR^{A5b}, —N(R^{A5c})₂, —C(═O)R^{A5d}, —C(═O)OR^{A5a}, —C(═O)N(R^{A5c})₂, —C(═NR^{A5c})R^{A5d}, —C(═NR^{A5c})OR^{A5a}, —C(═NR^{A5c})N(R^{A5c})₂, —S(O)₂R^{A5d}, —S(O)R^{A5d}, or two R^{A5} groups adjacent to each other are joined to form a 5- to 6-membered aryl, heteroaryl, heterocyclic or carbocyclic ring, wherein R^{A5a} is hydrogen, an optionally substituted aliphatic, optionally substituted heteroaliphatic, optionally substituted aryl, optionally substituted heteroaryl or a suitable hydroxyl protecting group; wherein R^{A5b} is hydrogen, an optionally substituted aliphatic, optionally substituted heteroaliphatic, optionally substituted aryl, optionally substituted heteroaryl or a suitable thiol protecting group; wherein each R^{A5c} is, independently, hydrogen, an optionally substituted aliphatic, optionally substituted heteroaliphatic, optionally substituted aryl, optionally substituted heteroaryl or a suitable amino protecting group, or two R^{A5c} groups are joined together to form a heterocyclic or heteroaryl group; and wherein each R^{A5d} is, independently, an optionally substituted aliphatic, optionally substituted heteroaliphatic, optionally substituted aryl, or an optionally substituted heteroaryl group, and

p is and integer between 0 to 5, inclusive.

In certain embodiments, -L- is —C(═O)—.

In certain embodiments, -L- is —C(═O)O—.

In certain embodiments, -L- is —C(═O)N(R^{e3})—.

In certain embodiments, -L- is —C(═NR^{e3})—.

In certain embodiments, -L- is —C(═NR^{e3})O—.

In certain embodiments, -L- is —C(═NR^{e3})N(R^{e3})—.

In certain embodiments, -L- is —S(O)₂—.

In certain embodiments, -L- is —S(O)—.

In certain embodiments, the group provided by Z, L and R^{L1} is of the formulae:

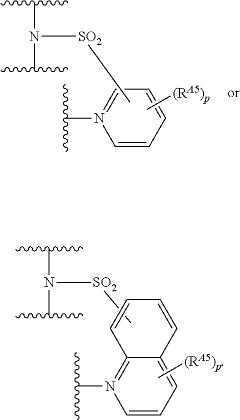

In certain embodiments, the group provided by Z, L and R^{L1} is of the formulae:

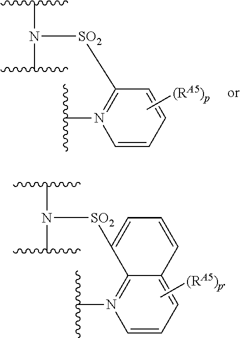

In certain embodiments, the group provided by Z, L and R^{L1} is:

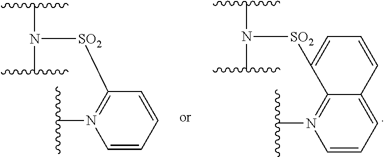

Group Z

In certain embodiments, Z is not linked to the ligand R^{L1} as in the case of a palladium (II) complex with a bidentate ligand. As defined generally above, in certain embodiments, Z is a bond, —O—, —S—, —C(R^{d})₂, —C(R^{d})═C(R^{d})—, —C(R^{d})═N—, or —N(R^{e})—;

wherein each instance of R^{d} is, independently, hydrogen, or an optionally substituted aliphatic, optionally substituted heteroaliphatic, optionally substituted aryl, or optionally substituted heteroaryl group; and

each instance of R^{e} is, independently, hydrogen, an optionally substituted aliphatic, optionally substituted heteroaliphatic, optionally substituted aryl, optionally substituted heteroaryl, —C(═O)R^{e1}, —C(═O)OR^{e2}, —C(═O)N(R^{e3})₂, —C(═NR^{e3})R^{e1}, —C(═NR^{e3})OR^{e2}, —C(═NR^{e3})N(R^{e3})₂, —S(O)₂R^{e1}, —S(O)R^{e1}, or a suitable amino protecting group, wherein R^{e1} is an optionally substituted aliphatic, optionally substituted heteroaliphatic, optionally substituted aryl or optionally substituted heteroaryl group; wherein R^{e2} is an optionally substituted aliphatic, optionally substituted heteroaliphatic, optionally substituted aryl, optionally substituted heteroaryl group, or a suitable hydroxyl protecting group; wherein R^{e3} is an optionally substituted aliphatic, optionally substituted heteroaliphatic, optionally substituted aryl, optionally substituted heteroaryl group, or a suitable amino protecting group, or two R^{e3} groups are joined to form an optionally substituted membered heterocyclic or heteroaryl ring.

In certain embodiments, Z is a bond.

In certain embodiments, Z is —C(R^{d})₂—. In certain embodiments, Z is —CH₂—.

In certain embodiments, Z is —C(R^{d})═C(R^{d})—. In certain embodiments, Z is —CH═CH—.

In certain embodiments, Z is —C(R^{d})═N—. In certain embodiments, Z is —CH═N—

In certain embodiments, Z is —O—.

In certain embodiments, Z is —S—.

In certain embodiments, Z is —NR^{e}—.

In certain embodiments, wherein Z is —NR^{e}—, the R^{e} group is of the formula —S(O)₂R^{e1}, wherein R^{e1} is an optionally substituted aliphatic, optionally substituted heteroaliphatic, optionally substituted aryl or optionally substituted heteroaryl group. In certain embodiments, the R^{e} group is of the formula —S(O)₂R^{e1}, wherein R^{e1} is an optionally substituted aryl or optionally substituted heteroaryl group. In certain embodiments, the R^{e} group is of the formula —S(O)₂R^{e1}, wherein R^{e1} is an optionally substituted heteroaryl group. In certain embodiments, the R^{e} group is of the formula —S(O)₂R^{e1}, wherein R^{e1} is an optionally substituted aryl group.

Exemplary —S(O)₂R^{e1} groups include, but are not limited to:

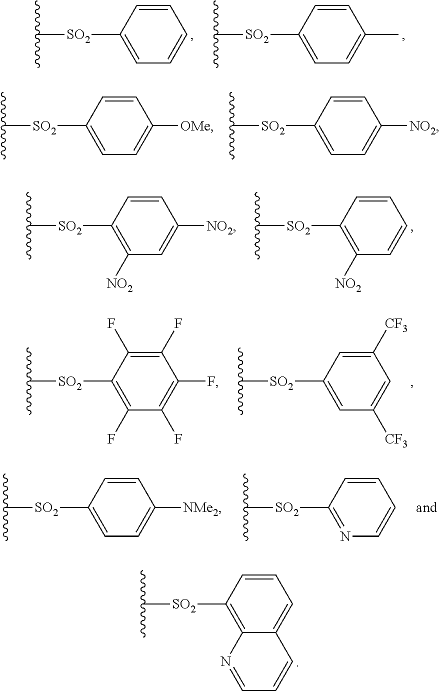

In certain embodiments, Z is of the formula:

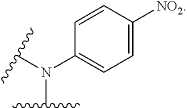

In certain embodiments, Z is of the formula:

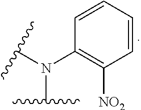

In certain embodiments, Z is of the formula:

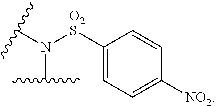

In certain embodiments, Z is of the formula:

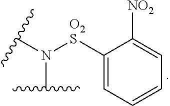

Exemplary Palladium(II) Complexes

In certain embodiments, the palladium(II) complex is selected from any of the following complexes:

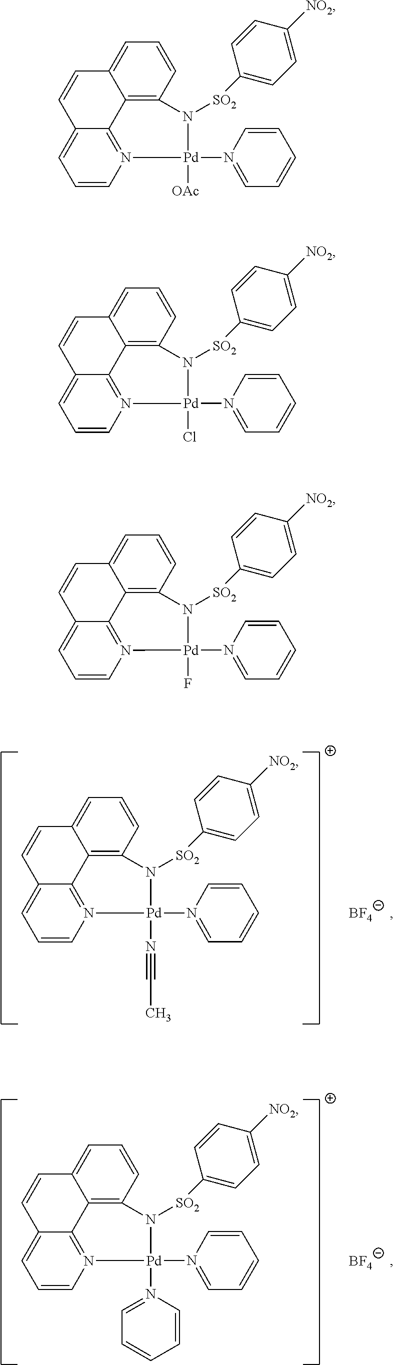

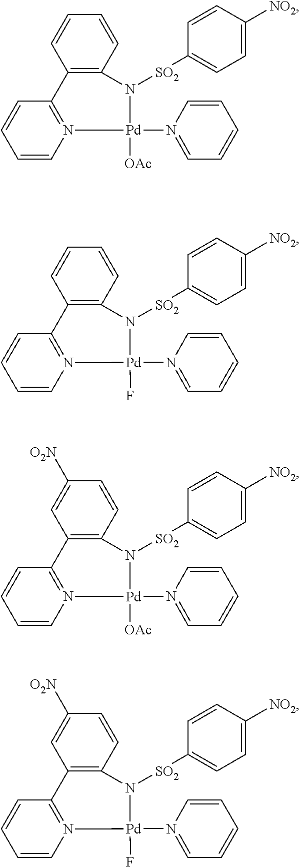

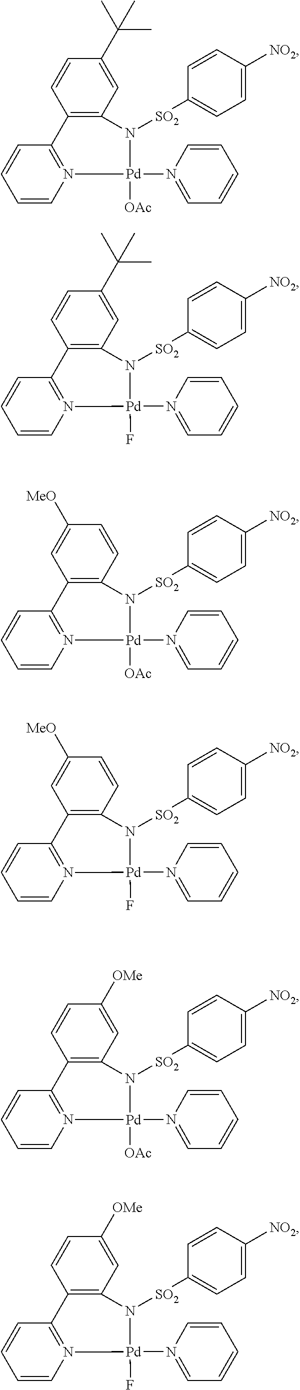

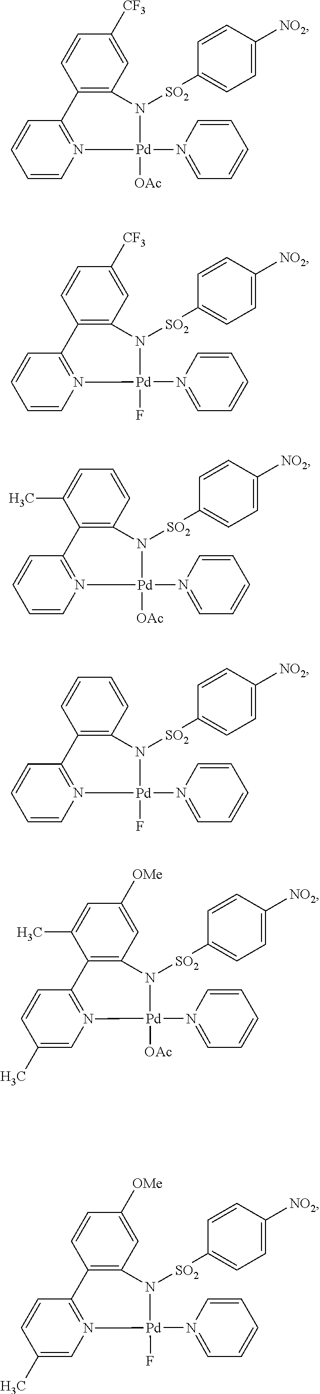

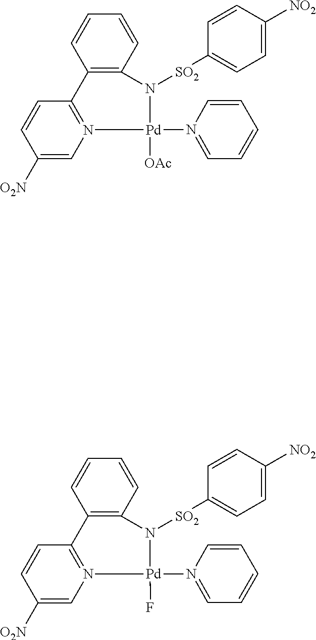

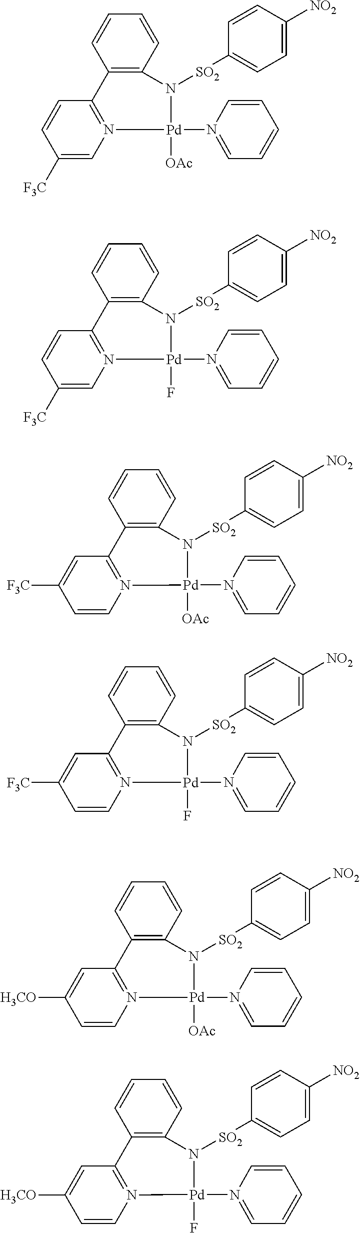

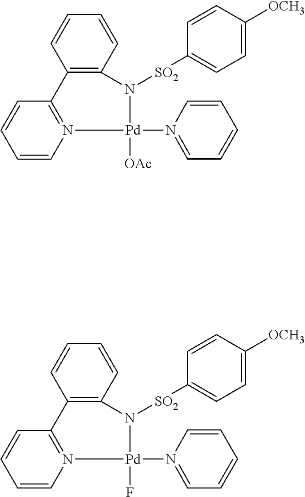

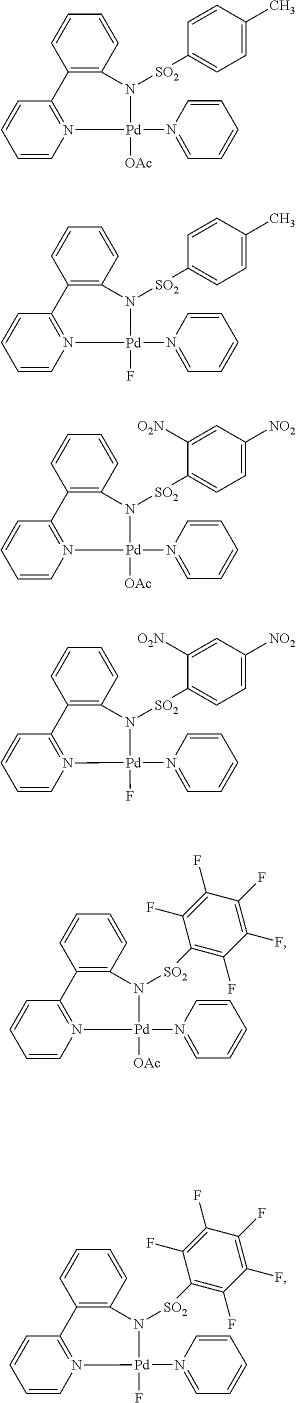

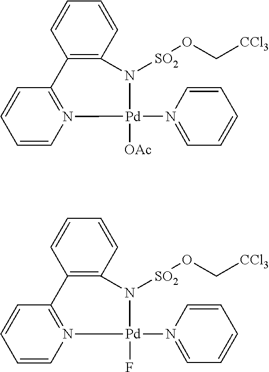

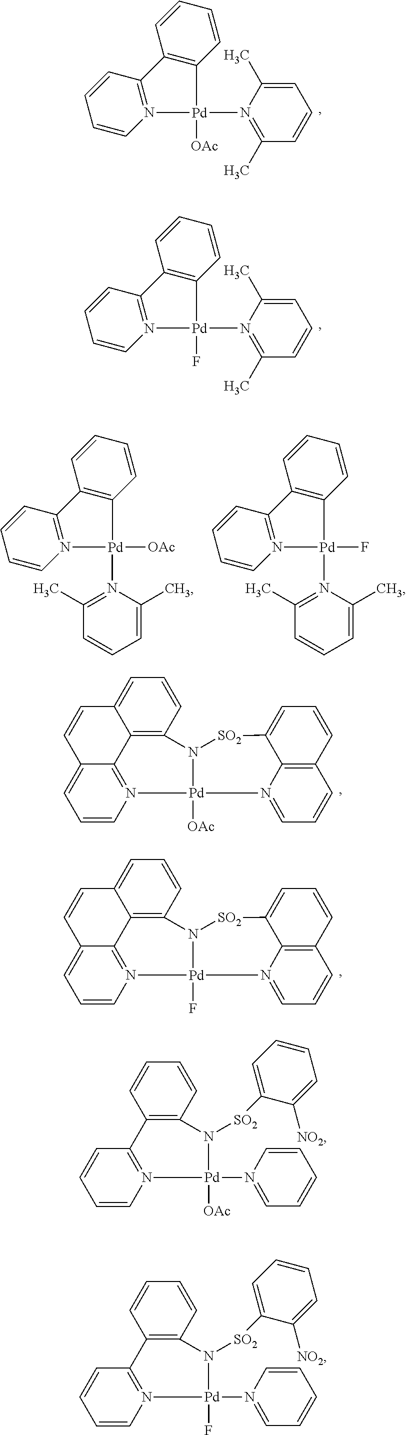

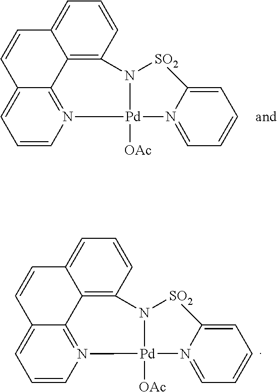

In certain embodiments, the palladium (II) complex:

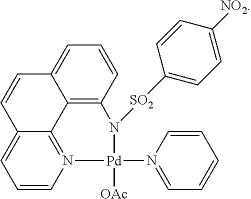

In certain embodiments, the palladium(II) complex is of the formula:

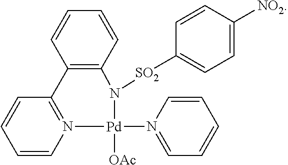

In certain embodiments, the palladium(II) complex is of the formula:

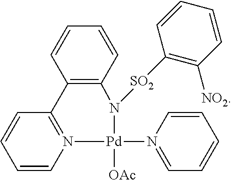

In certain embodiments, the palladium(II) complex is of the formula:

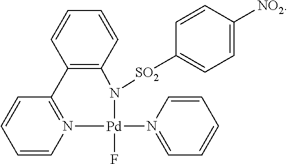

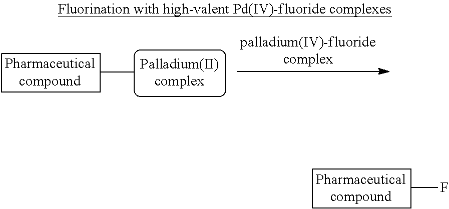

Upon reaction of an organopalladium(II) complex with a high-valent Pd(IV)-fluoride complex, the method provides a fluorinated organic compound in which the organic compound is fluorinated at the position at which it was bound to the palladium(II) center. In some embodiments, the organic compound is attached to the palladium(II) center (and subsequently fluorinated) via an aryl or heteroaryl moiety. For example, see Scheme 7.

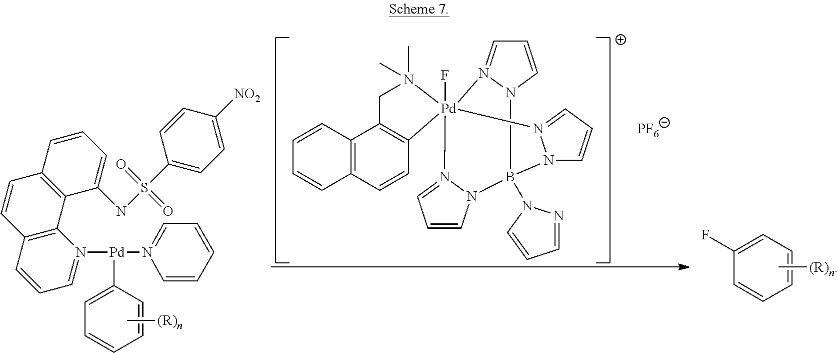

Exemplary methods of fluorinating a compound using a Pd(IV) complex are described in PCT Application PCT/US2009/046401, filed Jun. 5, 2009, which is incorporated herein by reference in its entirety.

Palladium (IV) Complexes

In certain embodiments, the complex is a Pd (IV) complex. Typically, the complex comprises one or more bidentate or tridentate ligands. Such ligands, particularly “scorpionate ligands,” are thought to stabilize the octahedral coordination sphere of the palladium (IV) center and thus prevent reductive elimination or other reductive pathways from an octahedral d⁶ palladium (IV) to a square planar d⁸ palladium (II).

In certain embodiments, the inventive high-valent palladium fluoride complex is of the formula:

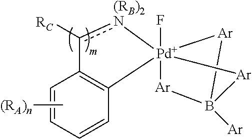

wherein

the dashed line represents the presence or absence of a bond;

Pd is palladium of a valency of +4;

n is an integer between 0 and 4, inclusive;

m is an integer between 0 and 3, inclusive;

each occurrence of Ar is a substituted or unsubstituted heteroaryl moiety;

each occurrence of R_{A} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR′; —C(═O)R′; —CO₂R′; —CN; —SCN; —SR′; —SOR′; —SO₂R′; —NO₂; —N(R′)₂; —NHC(O)R′; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R_{B} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR″; —C(═O)R″; —CO₂R″; —CN; —SCN; —SR″; —SOR″; —SO₂R″; —NO₂; —N(R″)₂; —NHC(O)R″; or —C(R″)₃; wherein each occurrence of R″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R_{C} is independently hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; wherein R_{B} and R_{C} may be taken together to form a cyclic structure; and a counteranion.

In certain embodiments, the high-valent palladium fluoride complex is of the formula:

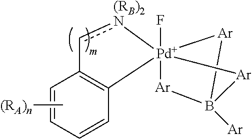

wherein

the dashed line represents the presence or absence of a bond;

Pd is palladium of a valency of +4;

n is an integer between 0 and 4, inclusive;

m is an integer between 0 and 3, inclusive;

each occurrence of Ar is a substituted or unsubstituted heteroaryl moiety;

each occurrence of R_{A} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR′; —C(═O)R′; —CO₂R′; —CN; —SCN; —SR′; —SOR′; —SO₂R′; —NO₂; —N(R′)₂; —NHC(O)R′; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R_{B} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR″; —C(═O)R″; —CO₂R″; —CN; —SCN; —SR″; —SOR″; —SO₂R″; —NO₂; —N(R″)₂; —NHC(O)R″; or —C(R″)₃; wherein each occurrence of R″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and a counteranion.

The counteranion may be any suitable anion. In certain embodiments, the counteranion has a charge of −1. In certain embodiments, the counteranion has a charge of −2. In certain embodiments, the counteranion has a charge of −3. The counteranion may be an organic or inorganic anion. In certain embodiments, the counteranion is an inorganic anion such as phosphate, chloride, bromide, iodide, etc. In other embodiments, the counteranion is an organic anion such as a carboxylic acid, sulfonate, phosphonate, borate, etc. In certain embodiments, the counteranion is triflate. In certain embodiments, the counteranion is tosylate. In certain embodiments, the counteranion is mesylate. In certain embodiments, the counteranion is hexafluorophosphate. In certain embodiments, the counteranion is tetraphenylborate. In certain embodiments, the counteranion is tetrafluoroborate. In certain embodiments, the counteranion is hexafluoroanimonate, [B[3,5-(CF₃)₂C₆H₃]₄]⁻, commonly abbreviated as [BAr^{F}₄]⁻.

In certain embodiments, n is 0, in which case the phenyl ring is unsubstituted. In certain embodiments, n is 1. In certain embodiments, n is 2. In certain embodiments, n is 3. In certain embodiments, n is 4. For the case where n is 1 or more, the substituents on the phenyl ring may have any substitution pattern.

In certain embodiments, m is 0. In certain embodiments, m is 1. In certain embodiments, m is 2. In certain embodiments, m is 3.

In certain embodiments, the dashed line represents a bond, thus forming an imine moiety. In other embodiments, the dashed line represents the absence of a bond resulting in only a single bond between the carbon atom and nitrogen atom.

In certain embodiments, at least one R_{A} is halogen. In certain embodiments, at least one occurrence of R_{A} is aliphatic. In certain embodiments, at least one occurrence of R_{A} is C₁-C₆alkyl. In certain embodiments, at least one occurrence of R_{A} is methyl. In certain embodiments, at least one occurrence of R_{A} is ethyl. In certain embodiments, at least one occurrence of R_{A} is propyl. In certain embodiments, at least one occurrence of R_{A} is butyl. In certain embodiments, at least one occurrence of R_{A} is heteroaliphatic. In certain embodiments, at least one occurrence of R_{A} is acyl. In certain embodiments, at least one occurrence of R_{A} is aryl. In certain embodiments, at least one occurrence of R_{A} is heteroaryl. In certain embodiments, at least one occurrence of R_{A} is —OR′. In certain embodiments, at least one occurrence of R_{A} is —N(R′)₂. In certain embodiments, at least one occurrence of R_{A} is —SR′. In certain embodiments, at least one occurrence of R_{A} is —NO₂. In certain embodiments, at least one occurrence of R_{A} is —CN. In certain embodiments, at least one occurrence of R_{A} is —SCN.

In certain embodiments, two occurrences of R_{A} taken together form a cyclic moiety. Such a cyclic moeity may be carbocyclic or heterocyclic. In certain embodiments, the cyclic moiety is a substituted or unsubstituted phenyl moiety. In certain embodiments, the cyclic moiety is an unsubstituted phenyl moiety. In certain embodiments, the cyclic moiety is a substituted or unsubstituted heteroaryl moiety.

In certain embodiments, at least one occurrence of R_{B} is hydrogen. In certain embodiments, both R_{B} are hydrogen. In certain embodiments, at least one occurrence of R_{B} is aliphatic. In certain embodiments, both occurrences of R_{B} are aliphatic. In certain embodiments, both occurrences of R_{B} are C₁-C₆alkyl. In certain embodiments, both occurrences of R_{B} are methyl. In certain embodiments, both occurrences of R_{B} are ethyl. In certain embodiments, both occurrences of R_{B} are propyl. In certain embodiments, both occurrences of R_{B} are butyl. In certain embodiments, at least one occurrence of R_{B} is heteroaliphatic. In certain embodiments, both occurrences of R_{B} are heteroaliphatic. In certain embodiments, at least one occurrence of R_{B} is acyl. In certain embodiments, at least one occurrence of R_{B} is aryl. In certain embodiments, at least one occurrence of R_{B} is heteroaryl.

In certain embodiments, both R_{B} are the same. In certain embodiments, the two R_{B} are different.

In certain embodiments, both R_{B} are taken together to form a heterocyclic moiety. In certain embodiments, both R_{B} are taken together to form a 5-membered heterocyclic moiety. In certain embodiments, both R_{B} are taken together to form a 6-membered heterocyclic moiety. In certain embodiments, both R_{B} are taken together to form an optionally substituted heteroaryl moiety.

In certain embodiments, one R_{B} moiety is covalently attached to a methylene group connecting the phenyl ring to the N atom, thus forming a heterocyclic moiety. Such a heterocyclic moiety may be a heteroaryl moiety. For example, in certain embodiments, the heterocyclic moiety is a pyridinyl moiety.

In certain embodiments, R_{C} is hydrogen. In certain embodiments, R_{C} is aliphatic. In certain embodiments, R_{C} is C₁-C₆alkyl. In certain embodiments, R_{C} is methyl. In certain embodiments, R_{C} is ethyl. In certain embodiments, R_{C} is propyl. In certain embodiments, R_{C} is butyl. In certain embodiments, R_{C} is heteroaliphatic. In certain embodiments, R_{C} is heteroaliphatic. In certain embodiments, R_{C} is acyl. In certain embodiments, R_{C} is aryl. In certain embodiments, R_{C} is heteroaryl. In certain embodiments, one R_{B} and R_{C} are taken together to form a heterocyclic moiety. In certain embodiments, one R_{B} and R_{C} are taken together to form a 5-membered heterocyclic moiety. In certain embodiments, one R_{B} and R_{C} are taken together to form a 6-membered heterocyclic moiety. In certain embodiments, one R_{B} and R_{C} are taken together to form an optionally substituted heteroaryl moiety.

In certain embodiments, Ar represents an optionally substituted heteroaryl moiety. In certain embodiments, at least one Ar is an unsubstituted heteroaryl moiety. In certain embodiments, all Ar are unsubstituted heteroaryl moieties. In certain embodiments, all Ar are optionally substituted 5-membered heteroaryl moieties. In certain embodiments, all Ar are nitrogen-containing 5-membered heteroaryl moieties, which are optionally substituted. In certain embodiments, all Ar are optionally substituted pyraolyl moieties. In certain embodiments, all Ar are optionally substituted imidazolyl moieties. In certain embodiments, all Ar are optionally substituted pyrrolyl moieties. In certain embodiments, all Ar are optionally substituted thiazolyl moieties. In certain embodiments, all Ar are optionally substituted oxazolyl moieties. In certain embodiments, all Ar are optionally substituted 6-membered heteroaryl moieties. In certain embodiments, all Ar are nitrogen-containing 6-membered heteroaryl moieties, which are optionally substituted. In certain embodiments, all Ar are optionally substituted pyridinyl moieties. In certain embodiments, all Ar are optionally substituted pyrazinyl moieties. In certain embodiments, all Ar are optionally substituted pyrimidinyl moieties. In certain embodiments, all Ar are optionally substituted pyridazinyl moieties. In certain embodiments, all Ar of the borate ligand are the same. In other embodiments, all Ar of the borate ligand are not the same. For example, a combination of heterocycle may constitute the borate ligand. In certain embodiments, a combination of heteroaryl moieties may constitute the borate ligand.

In certain embodiments, the palladium complex comprises a bidentate ligand of one of the formulae:

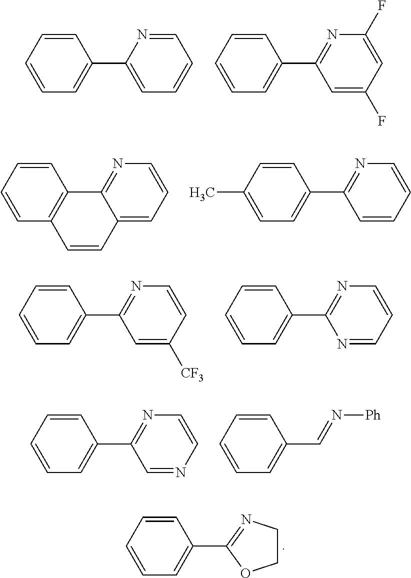

These ligands make a five-membered ring with the palladium atom with the nitrogen and a carbon coordinated to the central palladium.

In certain embodiments, the palladium complex is of the formula:

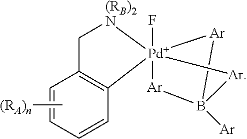

In certain embodiments, the palladium complex is of the formula:

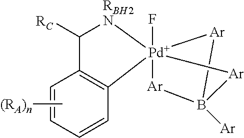

In certain embodiments, the palladium complex is of the formula:

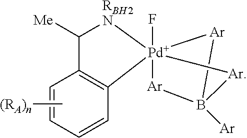

In certain embodiments, the palladium complex is of the formula:

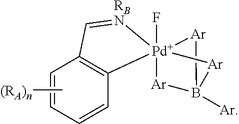

In certain embodiments, the palladium complex is of the formula:

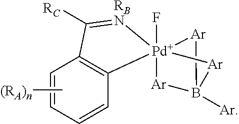

In certain embodiments, the palladium complex is of the formula:

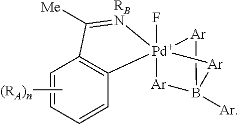

In certain embodiments, the palladium complex is of the formula:

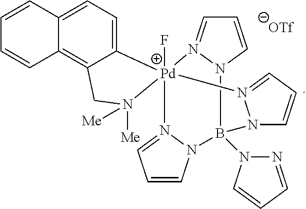

In certain embodiments, the palladium complex is of the formula:

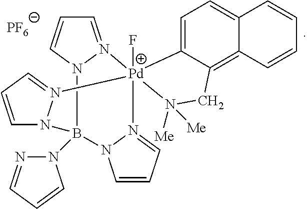

Preparation of High-Valent Palladium Fluoride Complexes

The inventive palladium complexes are typically prepared starting from disodium tetrachloropalladate. As would be appreciated by one of skill in the art, other palladium salts may also be used to prepare the inventive complexes. The starting material is subjected to cyclometallation to yield a palladium (II) chloride dimer. The chloride ligands are then substituted using the desired borate ligand to yield a palladium (II) borate, which is then oxidized with a fluorine-containing oxidizing reagent (e.g., 1-fluoro-pyridinium triflate, 2,4,6-trimethylpyridinium hexafluorophosphate, etc.) to yield the inventive palladium (IV) complex. An exemplary synthesis of a palladium (IV) fluoride complex is shown in FIG. 1.

In certain embodiments, the method of preparing an inventive palladium (IV) fluoride complex comprises (1) cyclometallating a palladium (II) salt with a bidentate ligand comprising a carbon-based with a carbon donor and a nitrogen donor to yield a palladium (II) chloride dimer; (2) reacting the palladium (II) dimer with a tridentate borate ligand under suitable conditions to yield a palladium (II) borate; and oxidizing the palladium (II) borate with a fluorinating reagent under suitable conditions to yield a palladium (IV) fluoride complex.

In certain embodiments, the bidentate ligand is of the formula:

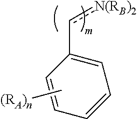

wherein

the dashed line represents the presence or absence of a bond;

n is an integer between 0 and 4, inclusive;

m is an integer between 0 and 3, inclusive;

each occurrence of R_{A} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR′; —C(═O)R′; —CO₂R′; —CN; —SCN; —SR′; —SOR′; —SO₂R′; —NO₂; —N(R′)₂; —NHC(O)R′; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

each occurrence of R_{B} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR″; —C(═O)R″; —CO₂R″; —CN; —SCN; —SR″; —SOR″; —SO₂R″; —NO₂; —N(R″)₂; —NHC(O)R″; or —C(R″)₃; wherein each occurrence of R″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety.

In certain embodiments, the borate ligand is tetrapyrazolylborate. In certain embodiments, the borate ligand is phenyltris(methimazolyl)borate.

In certain embodiments, an intermediate in the synthesis of a palladium (IV) fluoride complex is of the formula:

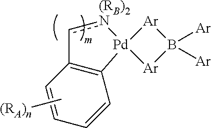

wherein

the dashed line represents the presence or absence of a bond;

Pd is palladium of a valency of +2;

n is an integer between 0 and 4, inclusive;

m is an integer between 0 and 3, inclusive;

each occurrence of Ar is a substituted or unsubstituted heteroaryl moiety;

each occurrence of R_{A} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR′; —C(═O)R′; —CO₂R′; —CN; —SCN; —SR′; —SOR′; —SO₂R′; —NO₂; —N(R′)₂; —NHC(O)R′; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R_{B} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR″; —C(═O)R″; —CO₂R″; —CN; —SCN; —SR″; —SOR″; —SO₂R″; —NO₂; —N(R″)₂; —NHC(O)R″; or —C(R″)₃; wherein each occurrence of R″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety.

In certain embodiments, n is 0, in which case the phenyl ring is unsubstituted. In certain embodiments, n is 1. In certain embodiments, n is 2. In certain embodiments, n is 3. In certain embodiments, n is 4. For the case where n is 1 or more, the substituents on the phenyl ring may have any substitution pattern.

In certain embodiments, m is 0. In certain embodiments, m is 1. In certain embodiments, m is 2. In certain embodiments, m is 3.

In certain embodiments, the dashed line represents a bond, thus forming an imine moiety. In other embodiments, the dashed line represents the absence of a bond resulting in only a single bond between the carbon atom and nitrogen atom.

In certain embodiments, at least one R_{A} is halogen. In certain embodiments, at least one occurrence of R_{A} is aliphatic. In certain embodiments, at least one occurrence of R_{A} is C₁-C₆alkyl. In certain embodiments, at least one occurrence of R_{A} is methyl. In certain embodiments, at least one occurrence of R_{A} is ethyl. In certain embodiments, at least one occurrence of R_{A} is propyl. In certain embodiments, at least one occurrence of R_{A} is butyl. In certain embodiments, at least one occurrence of R_{A} is heteroaliphatic. In certain embodiments, at least one occurrence of R_{A} is acyl. In certain embodiments, at least one occurrence of R_{A} is aryl. In certain embodiments, at least one occurrence of R_{A} is heteroaryl. In certain embodiments, at least one occurrence of R_{A} is —OR′. In certain embodiments, at least one occurrence of R_{A} is —N(R′)₂. In certain embodiments, at least one occurrence of R_{A} is —SR′. In certain embodiments, at least one occurrence of R_{A} is —NO₂. In certain embodiments, at least one occurrence of R_{A} is —CN. In certain embodiments, at least one occurrence of R_{A} is —SCN.

In certain embodiments, at least one occurrence of R_{B} is hydrogen. In certain embodiments, both R_{B} are hydrogen. In certain embodiments, at least one occurrence of R_{B} is aliphatic. In certain embodiments, both occurrences of R_{B} are aliphatic. In certain embodiments, both occurrences of R_{B} are C₁-C₆alkyl. In certain embodiments, both occurrences of R_{B} are methyl. In certain embodiments, both occurrences of R_{B} are ethyl. In certain embodiments, both occurrences of R_{B} are propyl. In certain embodiments, both occurrences of R_{B} are butyl. In certain embodiments, at least one occurrence of R_{B} is heteroaliphatic. In certain embodiments, both occurrences of R_{B} are heteroaliphatic. In certain embodiments, at least one occurrence of R_{B} is acyl. In certain embodiments, at least one occurrence of R_{B} is aryl. In certain embodiments, at least one occurrence of R_{B} is heteroaryl.

In certain embodiments, both R_{B} are the same. In certain embodiments, the two R_{B} are different.

In certain embodiments, both R_{B} are taken together to form a heterocyclic moiety. In certain embodiments, both R_{B} are taken together to form a 5-membered heterocyclic moiety. In certain embodiments, both R_{B} are taken together to form a 6-membered heterocyclic moiety. In certain embodiments, both R_{B} are taken together to form an optionally substituted heteroaryl moiety.

In certain embodiments, one R_{B} moiety is covalently attached to a methylene group connecting the phenyl ring to the N atom, thus forming a heterocyclic moiety. Such a heterocyclic moiety may be a heteroaryl moiety. For example, in certain embodiments, the heterocyclic moiety is a pyridinyl moiety.

In certain embodiments, Ar represents an optionally substituted heteroaryl moiety. In certain embodiments, at least one Ar is an unsubstituted heteroaryl moiety. In certain embodiments, all Ar are unsubstituted heteroaryl moieties. In certain embodiments, all Ar are optionally substituted 5-membered heteroaryl moieties. In certain embodiments, all Ar are nitrogen-containing 5-membered heteroaryl moieties, which are optionally substituted. In certain embodiments, all Ar are optionally substituted pyraolyl moieties. In certain embodiments, all Ar are optionally substituted imidazolyl moieties. In certain embodiments, all Ar are optionally substituted pyrrolyl moieties. In certain embodiments, all Ar are optionally substituted thiazolyl moieties. In certain embodiments, all Ar are optionally substituted oxazolyl moieties. In certain embodiments, all Ar are optionally substituted 6-membered heteroaryl moieties. In certain embodiments, all Ar are nitrogen-containing 6-membered heteroaryl moieties, which are optionally substituted. In certain embodiments, all Ar are optionally substituted pyridinyl moieties. In certain embodiments, all Ar are optionally substituted pyrazinyl moieties. In certain embodiments, all Ar are optionally substituted pyrimidinyl moieties. In certain embodiments, all Ar are optionally substituted pyridazinyl moieties. In certain embodiments, all Ar of the borate ligand are the same. In other embodiments, all Ar of the borate ligand are not the same. For example, a combination of heterocycle may constitute the borate ligand. In certain embodiments, a combination of heteroaryl moieties may constitute the borate ligand.

In certain embodiments, the intermediate is of the formula:

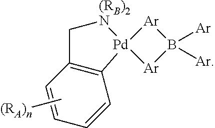

In certain embodiments, the intermediate is of the formula:

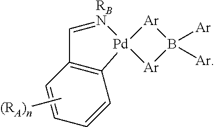

In certain embodiments, the intermediate is of the formula:

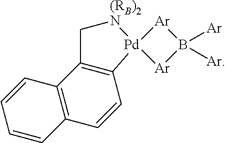

In certain embodiments, the intermediate is of the formula:

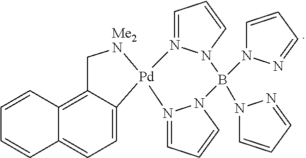

As can be appreciated by the skilled artisan, alternative methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Methods of Treatment

The compounds and compositions described herein can be administered to cells in culture, e.g. in vitro or ex vivo, or to a subject, e.g., in vivo, to treat, prevent, and/or diagnose a variety of disorders, including those described herein below.

As used herein, the term “treat” or “treatment” is defined as the application or administration of a compound, alone or in combination with, a second compound to a subject, e.g., a patient, or application or administration of the compound to an isolated tissue or cell, e.g., cell line, from a subject, e.g., a patient, who has a disorder (e.g., a disorder as described herein), a symptom of a disorder, or a predisposition toward a disorder, with the purpose to cure, heal, alleviate, relieve, alter, remedy, ameliorate, improve or affect the disorder, one or more symptoms of the disorder or the predisposition toward the disorder (e.g., to prevent at least one symptom of the disorder or to delay onset of at least one symptom of the disorder).

As used herein, an amount of a compound effective to treat a disorder, or a “therapeutically effective amount” refers to an amount of the compound which is effective, upon single or multiple dose administration to a subject, in treating a cell, or in curing, alleviating, relieving or improving a subject with a disorder beyond that expected in the absence of such treatment.

As used herein, an amount of a compound effective to prevent a disorder, or a “a prophylactically effective amount” of the compound refers to an amount effective, upon single- or multiple-dose administration to the subject, in preventing or delaying the occurrence of the onset or recurrence of a disorder or a symptom of the disorder.

As used herein, the term “subject” is intended to include human and non-human animals. Exemplary human subjects include a human patient having a disorder, e.g., a disorder described herein or a normal subject. The term “non-human animals” of the invention includes all vertebrates, e.g., non-mammals (such as chickens, amphibians, reptiles) and mammals, such as non-human primates, domesticated and/or agriculturally useful animals, e.g., sheep, dog, cat, cow, pig, etc.

Described herein are compounds and compositions useful as opioid analgesics, and also compounds used to treat opioid dependence, such as an opioid analgesic or opioid dependence agent described herein. In general, the compounds described herein are fluorinated derivatives of a pharmaceutical agent (e.g., an opioid receptor agonist). Also envisioned herein are other opioid analgesics and agents for treating opioid dependence, wherein one or more fluorine moieties have been added to the pharmaceutical agent, e.g., replacing a hydrogen or functional group such as an —OH with a fluorine.

Enkephalins

An enkephalin is a pentapeptide. Enkephalins are generally involved in regulating nociception in the body. The enkephalins are generally endogenous opioid ligands, e.g., endorphins, which can be internally derived and bind to the body's opioid receptors. Two naturally occurring forms of enkephalin are known, one containing leucine (“Leu”), and the other containing methionine (“Met”) at the C-terminus. Both are products of the proenkephalin gene and are shown below.

| | |
|---|---|
| Met-enkephalin: | Tyr-Gly-Gly-Phe-Met. |
| Leu-enkephalin: | Tyr-Gly-Gly-Phe-Leu. |

The enkephalins can act as natural painkillers. The receptors for enkephalins are the opioid receptors (delta receptor), for which other opioids serve as ligands as well.

Opioids

An opioid is a chemical substance that has a morphine-like action in the body. There are a number of broad classes of opioids, including, natural opiates (alkaloids contained in the resin of the opium poppy including morphine, codeine and thebaine), semi-synthetic opiates (created from the natural opioids), fully synthetic opioids, and endogenous opioid peptides (produced naturally in the body).

Opioids can be used for pain relief. These agents generally work by binding to opioid receptors, which are found principally in the central nervous system and the gastrointestinal tract. There are three principal classes of opioid receptors, μ, κ, δ, although up to seventeen have been reported, and include the ε, ι, λ, and ζ receptors. In addition, there are three subtypes of μ receptor: μ₁ and μ₂, and the newly discovered μ₃. Another receptor of clinical importance is the opioid-receptor-like receptor 1 (ORL1), which is involved in pain responses as well as having a major role in the development of tolerance to n-opioid agonists used as analgesics. These are all G-protein coupled receptors acting on GABAergic neurotransmission. The pharmacodynamic response to an opioid depends on which receptor it binds, its affinity for that receptor, and whether the opioid is an agonist or an antagonist. For example, the supraspinal analgesic properties of the opioid agonist morphine are mediated by activation of the μ₁ receptor, respiratory depression and physical dependence (dependency) by the μ₂ receptor, and sedation and spinal analgesia by the κ receptor. Each group of opioid receptors elicits a distinct set of neurological responses, with the receptor subtypes (such as μ₁ and μ₂ for example) providing even more specific responses. Unique to each opioid is their distinct binding affinity to the group(s) of opioid receptors (e.g., the μ, κ, and δ opioid receptors are activated at different magnitudes according to the specific receptor binding affinities of the opioid, such as the μ opioid receptor effects being the primary receptor response to the opioid morphine, or the κ opioid receptor residing as the primary binding receptor to ketazocine).

Clinical use of opioids include, e.g., Analgesia i.e. to combat pain of various types and induction and the continuance of anesthesia as well as allaying patient apprehension right before the procedure (Fentanyl, oxymorphone, hydromorphone, and morphine are commonly used for this purpose), Cough (codeine, dihydrocodeine, ethylmorphine (dionine), hydromorphone and hydrocodone, with morphine or methadone can be used for this purpose), Diarrhea (generally loperamide, difenoxin or diphenoxylate, but paregoric, powdered opium or laudanum or morphine may be used in some cases of severe diarrheal diseases), Diarrhea of Irritable Bowel Syndrome (e.g., Codeine, paregoric, diphenoxylate, difenoxin, loperamide, laudanum), Anxiety due to shortness of breath (e.g., oxymorphone and dihydrocodeine), and Detoxification (e.g., methadone and buprenorphine).

In some instances, where a subject has become dependent on an opioid, the subject is administered a compound to treat the opioid dependence. Opioid dependency is a medical diagnosis characterized by an individual's inability to stop using opioids even when objectively in his or her best interest to do so. In 1964 the WHO Expert Committee on Drug Dependence introduced “dependence” as “A cluster of physiological, behavioral and cognitive phenomena of variable intensity, in which the use of a psychoactive drug (or drugs) takes on a high priority. The necessary descriptive characteristics are preoccupation with a desire to obtain and take the drug and persistent drug-seeking behavior. Treatment approaches include abstinence-based and harm-reduction methodologies. Both include participation in detoxification through the use of methadone or other long-acting opioids. Alternative detox protocols call for total abstention from all opiates, with the use of various benzodiazepines and other medications to reduce the uncomfortable withdrawal symptoms associated with abstinence. In an abstinence-based approach, a gradual taper of the medications follows detox, while in the harm-reduction approach, the patient remains on an ongoing dose of methadone or buprenorphine.

Compositions and Routes of Administration

The compositions delineated herein include the compounds delineated herein (e.g., a compound described herein), as well as additional therapeutic agents if present, in amounts effective for achieving a modulation of disease or disease symptoms, including those described herein.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

In some embodiments, the composition can include a compound that slows the degradation of a peptide, e.g., a protease inhibitor.

In some embodiments, the composition includes a permeation enhancer, for example a component that improves the bioavailability of a compound described herein. Exemplary permeation enhancers include surfactants, fatty acids, polymers (e.g., positively charged amino acid polymers), lipids, and other compounds.

In some embodiments, the composition is formulated for nasal, rectal or parenteral administration (e.g., by way of a route that avoids the GI tract). Exemplary formulations include nebulized liquids, small particulate compositions, emulsions, waxes, solid suspensions, solutions, etc.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

Kits

A compound described herein described herein can be provided in a kit. The kit includes (a) a compound described herein, e.g., a composition that includes a compound described herein, and, optionally (b) informational material. The informational material can be descriptive, instructional, marketing or other material that relates to the methods described herein and/or the use of a compound described herein for the methods described herein.

The informational material of the kits is not limited in its form. In one embodiment, the informational material can include information about production of the compound, molecular weight of the compound, concentration, date of expiration, batch or production site information, and so forth. In one embodiment, the informational material relates to methods for administering the compound.

In one embodiment, the informational material can include instructions to administer a compound described herein in a suitable manner to perform the methods described herein, e.g., in a suitable dose, dosage form, or mode of administration (e.g., a dose, dosage form, or mode of administration described herein). In another embodiment, the informational material can include instructions to administer a compound described herein to a suitable subject, e.g., a human, e.g., a human having or at risk for a disorder described herein.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In addition to a compound described herein, the composition of the kit can include other ingredients, such as a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, and/or a second agent for treating a condition or disorder described herein. Alternatively, the other ingredients can be included in the kit, but in different compositions or containers than a compound described herein. In such embodiments, the kit can include instructions for admixing a compound described herein and the other ingredients, or for using a compound described herein together with the other ingredients.

In some embodiments, the components of the kit are stored under inert conditions (e.g., under Nitrogen or another inert gas such as Argon). In some embodiments, the components of the kit are stored under anhydrous conditions (e.g., with a desiccant). In some embodiments, the components are stored in a light blocking container such as an amber vial.

A compound described herein can be provided in any form, e.g., liquid, dried or lyophilized form. It is preferred that a compound described herein be substantially pure and/or sterile. When a compound described herein is provided in a liquid solution, the liquid solution preferably is an aqueous solution, with a sterile aqueous solution being preferred. When a compound described herein is provided as a dried form, reconstitution generally is by the addition of a suitable solvent. The solvent, e.g., sterile water or buffer, can optionally be provided in the kit.

The kit can include one or more containers for the composition containing a compound described herein. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

### EXAMPLES

### Example 1

### Preparation of Derivatives of Fluoro-leu-enkephalin

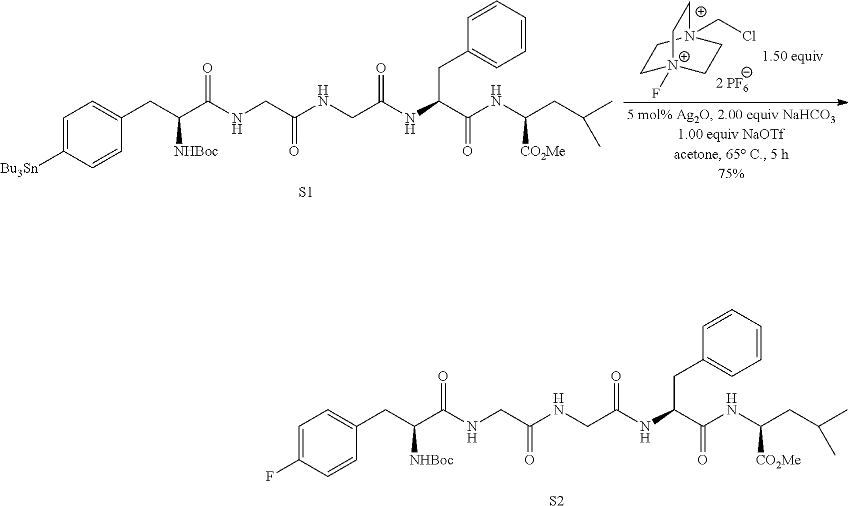

To S1 (9.43 mg, 0.010 mmol, 1.00 equiv) in acetone (0.2 mL) at 23° C. was added silver oxide (0.12 mg, 0.0005 mmol, 0.05 equiv), sodium bicarbonate (1.72 mg, 0.020 mmol, 2.00 equiv), sodium trifluoromethanesulfonate (1.74 mg, 0.010 mmol, 1.00 equiv) and 1-chloromethyl-4-fluoro-1,4-diazoniabicyclo[2.2.2]octane bis(hexafluorophosphate) (7.05 mg, 0.150 mmol, 1.50 equiv). The reaction mixture was stirred at 65° C. for 5 h in a sealed vial, then cooled to 23° C. The yield was determined to be 75%.

¹H NMR (500 MHz, CDCl₃, 23° C., δ): 7.42-7.00 (m, 9H), 6.95-6.90 (br s, 3H), 5.31 (br s, 1H), 4.60 (m, 1H), 4.42 (m, 1H), 4.22 (m, 1H), 4.00-3.70 (m, 4H), 3.60 (s, 3H), 3.20-3.00 (m, 3H), 2.90-2.80 (m, 1H).

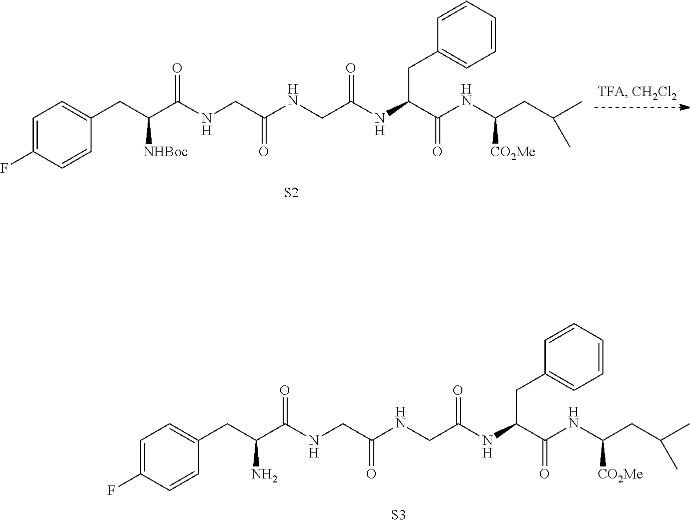

To S2 in CH₂Cl₂ at 0° C. will be added trifluoro acetic acid. The reaction mixture will be stirred for 2 hr at 0° C. The reaction mixture will be concentrated in vacuo and dissolved in CH₂Cl₂, washed with NaHCO₃(aq) and dried. The filtrate will be concentrated in vacuo and purified by chromatography on silica gel.

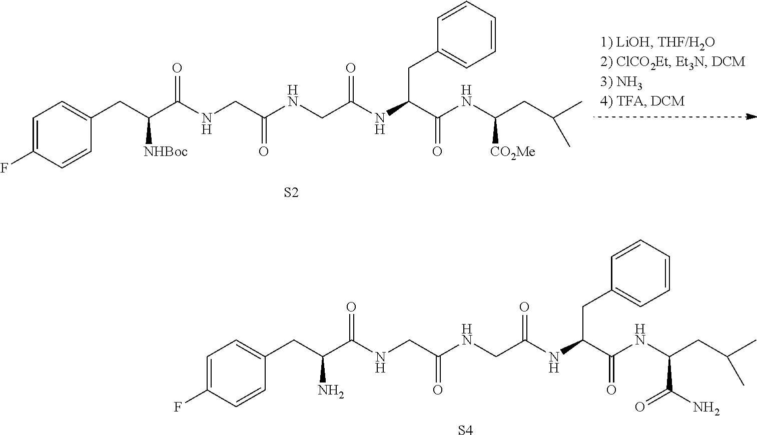

To S2 in THF at 0° C. will be added LiOH. The reaction mixture will be stirred for 2 hr at 0° C. The reaction mixture will be diluted with ethyl acetate and the pH will be adjusted to 2-3 by using HCl (1N). The layers will be separated and the aqueous layer will be extracted with ethyl acetate and dried (Na₂SO₄). The filtrate will be concentrated in vacuo.

To this product in THF at −10° C. will be added TEA and ClCO₂Et. The reaction mixture will be stirred for 30 min before a stream of anhydrous ammonia gas will be introduced. After 90 min the THF will be removed. Water will be added, the mixture will be extracted with Et₂O. Then the solvent will be removed.

To this product in CH₂Cl₂ at 0° C. will be added trifluoro acetic acid. The reaction mixture will be stirred for 2 hr at 0° C. The reaction mixture will be concentrated in vacuo and dissolved in CH₂Cl₂, washed with NaHCO₃(aq) and dried. The filtrate will be concentrated in vacuo and purified by HPLC to afford product S4.

### Example 2

### Preparation of a Derivative of Fluoro-leu-enkephalin

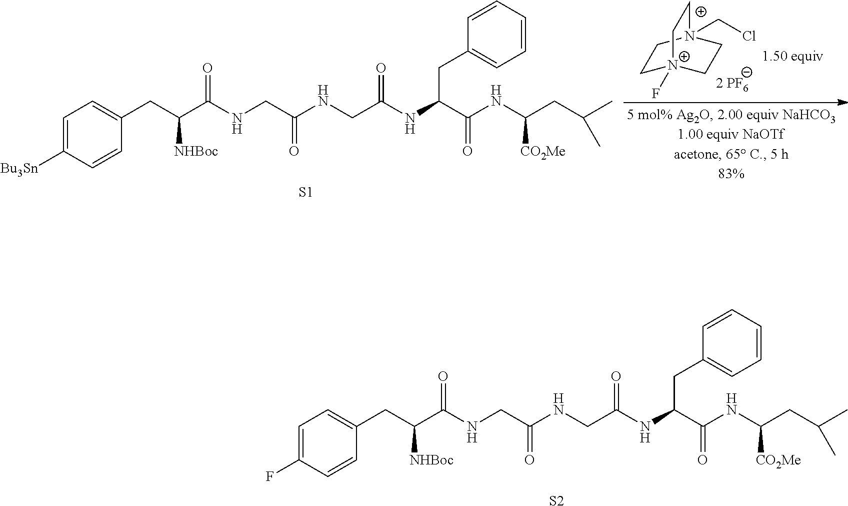

To S1 (9.43 mg, 0.010 mmol, 1.00 equiv) in acetone (0.2 mL) at 23° C. was added silver oxide (0.12 mg, 0.0005 mmol, 0.05 equiv), sodium bicarbonate (1.72 mg, 0.020 mmol, 2.00 equiv), sodium trifluoromethanesulfonate (1.74 mg, 0.010 mmol, 1.00 equiv) and 1-chloromethyl-4-fluoro-1,4-diazoniabicyclo[2.2.2]octane bis(hexafluorophosphate) (7.05 mg, 0.150 mmol, 1.50 equiv). The reaction mixture was stirred at 65° C. for 5 h in a sealed vial, then cooled to 23° C. The yield was determined to be 83%.

¹H NMR (500 MHz, CDCl₃, 23° C., δ): 7.42-7.00 (m, 9H), 6.95-6.90 (br s, 3H), 5.31 (br s, 1H), 4.60 (m, 1H), 4.42 (m, 1H), 4.22 (m, 1H), 4.00-3.70 (m, 4H), 3.60 (s, 3H), 3.20-3.00 (m, 3H), 2.90-2.80 (m, 1H).

What is claimed is:

1. A compound of the following formula: 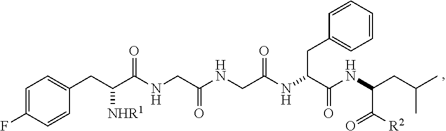
wherein:
R¹ is hydrogen, alkyl or PG¹;
R² is hydroxy, alkoxy, aryloxy, arylalkoxy, —O(PG²), amino, alkylamino, dialkylamino, or —NH(PG¹);
PG¹ is an amino protecting group; and
PG² is a hydroxy protecting group,
or a pharmaceutically acceptable salt thereof.

2. The compound of claim 1, wherein R¹ is hydrogen.

3. The compound of claim 1, wherein R¹ is PG¹.

4. The compound of claim 3, wherein PG¹ is tert-butyloxycarbonyl.

5. The compound of claim 1, wherein R² is hydroxy.

6. The compound of claim 1, wherein R² is alkoxy.

7. The compound of claim 6, wherein R² is methoxy.

8. The compound of claim 1, wherein R² is amino.

9. The compound of claim 1, wherein the fluorine substituent comprises F¹⁸.

10. The compound of claim 1, wherein the fluorine substituent comprises F¹⁹.

11. A compound of the following formula: 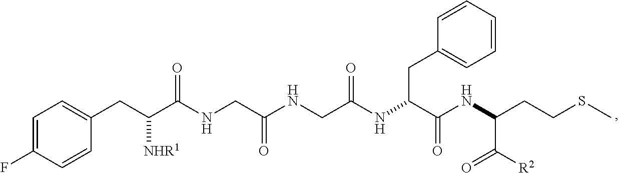
wherein:
R¹ is hydrogen, alkyl or PG¹;
R² is hydroxy, alkoxy, aryloxy, arylalkoxy, —O(PG²), amino, alkylamino, dialkylamino, or —NH(PG¹);
PG¹ is an amino protecting group; and
PG² is a hydroxy protecting group,
or a pharmaceutically acceptable salt thereof.

12. The compound of claim 11, wherein R¹ is hydrogen.

13. The compound of claim 11, wherein R¹ is PG¹.

14. The compound of claim 13, wherein PG¹ is tert-butyloxycarbonyl.

15. The compound of claim 11, wherein R² is hydroxy.

16. The compound of claim 11, wherein R² is alkoxy.

17. The compound of claim 16, wherein R² is methoxy.

18. The compound of claim 11, wherein R² is amino.

19. A compound selected from the group consisting of:

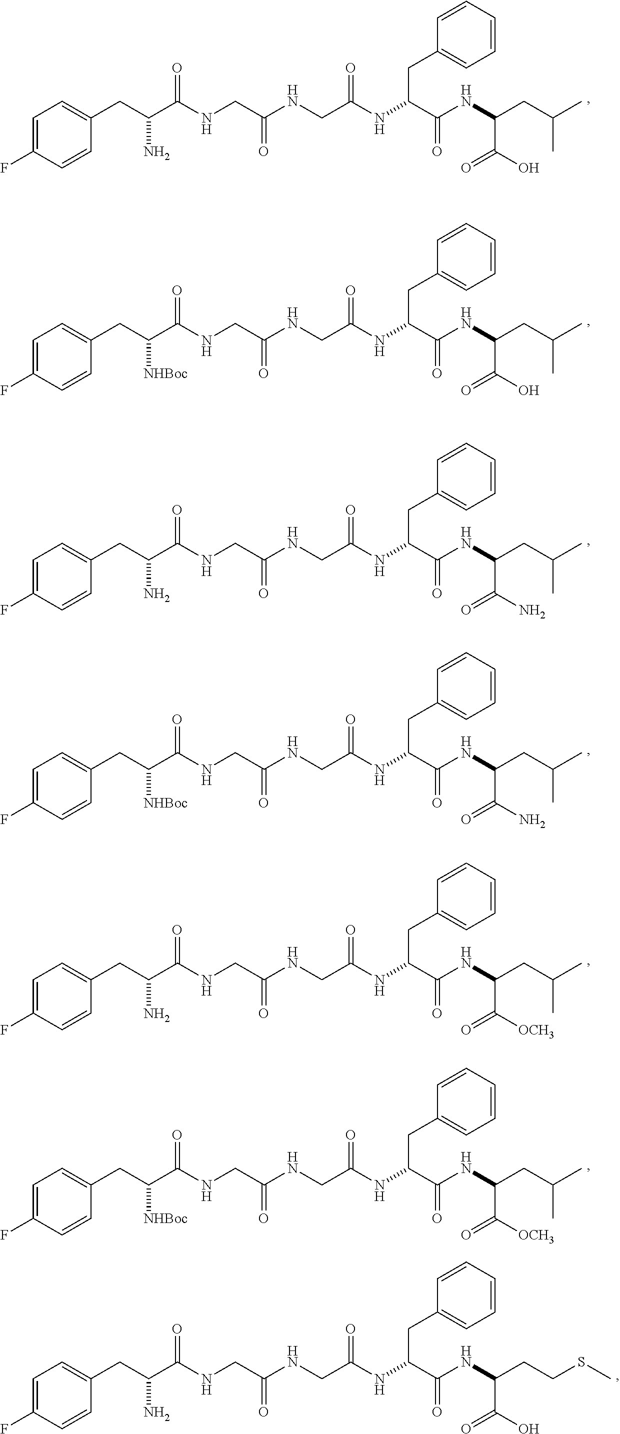

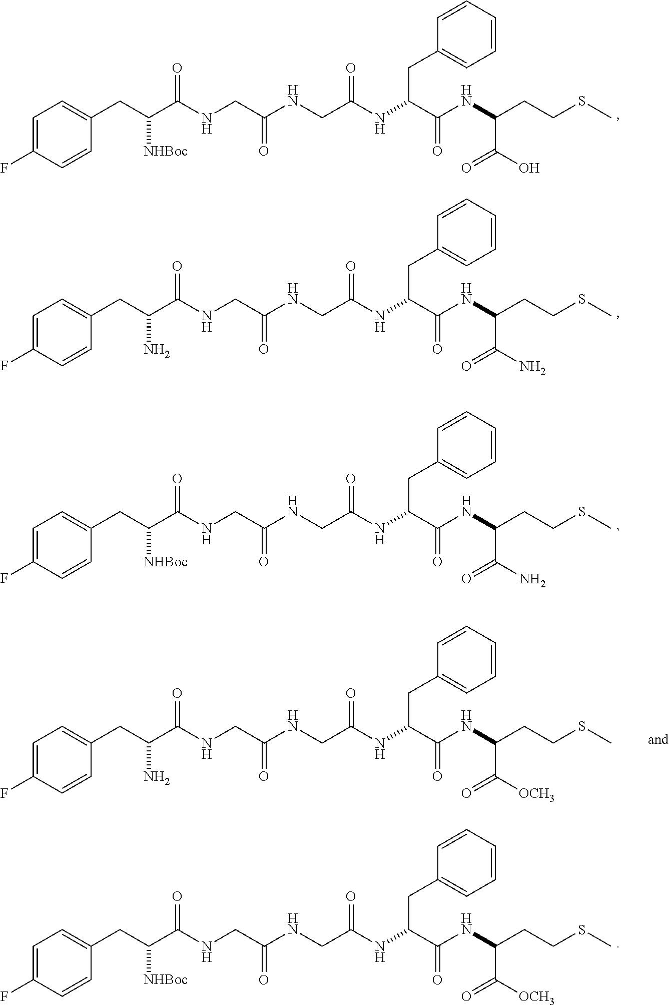

20. A composition comprising a compound of claim 1.

21. The composition of claim 20, wherein the composition is a pharmaceutical composition.

22. A kit comprising a compound of claim 1 or a composition of claim 20.

23. A method of treating or preventing a disorder, the method comprising administering a compound of claim 1 or a composition of claim 20 to a subject.

24. The method of claim 23, wherein the disorder is a disorder that can be treated with an opioid analgesic.

25. The method of claim 23, wherein the disorder is an opioid dependence disorder.
